(19) United States
(12) Patent Application Publication
(10) Pub. No.: US 2024/0423516 A1
(43) Pub. Date: Dec. 26, 2024

(54) Dressing System

(71) Applicant: Solventum Intellectual Properties Company, Maplewood, MN (US)

(72) Inventors: Li YU, Santa Clara, CA (US); Dawn V. MUYRES, Stillwater, MN (US); Mark A. ROEHRIG, Stillwater, MN (US); John A. WHEATLEY, Stillwater, MN (US); Jason W. BJORK, Cottage Grove, MN (US); Przemyslaw P. MARKOWICZ, Woodbury, MN (US)

(73) Assignee: Solventum Intellectual Properties Company, Maplewood, MN (US)

(21) Appl. No.: 18/683,644

(22) PCT Filed: Jul. 29, 2022
(86) PCT No.: PCT/IB2022/057089

§ 371 Date: Feb. 14, 2024

Related U.S. Application Data
(60) Provisional application No. 63/234,285, filed on Aug. 18, 2021.

(51) Int. Cl. A61B 5/1455; A61B 5/00

(52) U.S. Cl. CPC A61B 5/1455; A61B 5/6833

(57) ABSTRACT

A dressing system for sensing a presence of an analyte includes a first layer, and a second layer facing a first major surface of the first layer. The second layer has a second permeability to the analyte less than a first permeability of the first layer. The dressing system further includes a first fiber configured to deliver an excitation light. The dressing system further includes at least one sensor layer including a sensor material configured to receive the excitation light from the first fiber and emit an emitted light in response to the excitation light. The dressing system further includes a second fiber separate from the first fiber and configured to receive the emitted light from the at least one sensor layer.

### TECHNICAL FIELD

The present disclosure relates, in general, to a dressing system. In particular, the present disclosure relates to a dressing system for sensing a presence of an analyte.

### BACKGROUND

Sensing and monitoring certain analytes may be required in various applications. For example, sensing and monitoring of an oxygen concentration underneath a dressing may be required to determine if the oxygen concentration is adequate to allow optimal cellular function and wound healing of skin and tissues.

### SUMMARY

In a first aspect, the present disclosure provides a dressing system for sensing a presence of an analyte. The dressing system includes a first layer including a first major surface and a second major surface opposite to the first major surface. The first layer has a first permeability to the analyte. The dressing system further includes a second layer facing the first major surface of the first layer. The second layer has a second permeability to the analyte less than the first permeability of the first layer. The dressing system further includes a first fiber at least partially disposed between the first layer and the second layer. The first fiber is configured to deliver an excitation light. The dressing system further includes at least one sensor layer including a sensor material disposed between the first layer and the second layer. The at least one sensor layer is configured to receive the excitation light from the first fiber and emit an emitted light in response to the excitation light. The emitted light includes a first optical property sensitive to the presence of the analyte. The dressing system further includes a second fiber separate from the first fiber and disposed between the first layer and the second layer. The second fiber is configured to receive the emitted light from the at least one sensor layer.

### BRIEF DESCRIPTION OF THE DRAWINGS

Exemplary embodiments disclosed herein is more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labelled with the same number.

FIG. 1 illustrates a schematic exploded perspective view of a dressing system for sensing a presence of an analyte according to an embodiment of the present disclosure;

FIG. 2A illustrates a graph depicting optical transmittance percentage versus wavelength of an optical filter of the dressing system of FIG. 1 according to an embodiment of the present disclosure;

FIG. 2B illustrates a graph depicting optical reflectance percentage versus wavelength of a first layer and a second layer of the dressing system of FIG. 1 according to an embodiment of the present disclosure;

FIG. 3 illustrates a schematic top view of a dressing system for sensing a presence of an analyte according to an embodiment of the present disclosure;

FIG. 4 illustrates a schematic top view of a dressing system for sensing a presence of an analyte according to an embodiment of the present disclosure;

FIG. 5 illustrates a schematic top view of a dressing system for sensing a presence of an analyte according to an embodiment of the present disclosure;

FIG. 6 illustrates a schematic top view of a dressing system for sensing a presence of an analyte according to an embodiment of the present disclosure;

FIG. 7 illustrates a schematic top view of a dressing system for sensing a presence of an analyte according to an embodiment of the present disclosure;

FIG. 8 illustrates a schematic top view of a dressing system for sensing a presence of an analyte according to an embodiment of the present disclosure;

FIG. 9 illustrates a schematic exploded perspective view of a dressing system for sensing a presence of an analyte according to an embodiment of the present disclosure;

FIG. 10A illustrates a schematic top view of a first fiber of the dressing system of FIG. 9 according to an embodiment of the present disclosure;

FIG. 10B illustrates a schematic top view of a second fiber of the dressing system of FIG. 9 according to an embodiment of the present disclosure;

FIG. 11 illustrates a graph depicting optical reflectance percentage versus wavelength of a first reflective layer and a second reflective layer of the dressing system of FIG. 9 according to an embodiment of the present disclosure;

FIG. 12 illustrates a schematic top view of a dressing system for sensing a presence of an analyte according to an embodiment of the present disclosure;

FIG. 13 illustrates a schematic top view of a dressing system for sensing a presence of an analyte according to an embodiment of the present disclosure;

FIG. 14 illustrates a schematic top view of a dressing system for sensing a presence of an analyte according to an embodiment of the present disclosure; and

FIG. 15 illustrate a schematic block diagram of a dressing system for sensing a presence of an analyte according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and is made without departing from the scope or spirit of the present disclosure. The following detailed description, therefore, is not to be taken in a limiting sense.

As used herein, “at least one of A and B” should be understood to mean “only A, only B, or both A and B”.

Oxygen may be crucial for wound healing and may be required during wound repair for a host of processes including epithelial cell migration, collagen synthesis, fibroblast proliferation, and neutrophil influx to reduce infection. An adequate oxygen concentration underneath a dressing may be essential for cellular function and wound healing of skin and tissues. Monitoring oxygen concentration underneath the dressing may be important to avoid ischemia or necrosis. Therefore, monitoring and sensing tissue oxygen concentration may be useful in clinical assessment and decision-making.

The present disclosure provides a dressing system for sensing a presence of an analyte. The dressing system includes a first layer including a first major surface and a second major surface opposite to the first major surface. The first layer has a first permeability to the analyte. The dressing system further includes a second layer facing the first major surface of the first layer. The second layer has a second permeability to the analyte less than the first permeability of the first layer. The dressing system further includes a first fiber at least partially disposed between the first layer and the second layer. The first fiber is configured to deliver an excitation light. The dressing system further includes at least one sensor layer including a sensor material disposed between the first layer and the second layer. The at least one sensor layer is configured to receive the excitation light from the first fiber and emit an emitted light in response to the excitation light. The emitted light includes a first optical property sensitive to the presence of the analyte. The dressing system further includes a second fiber separate from the first fiber and disposed between the first layer and the second layer. The second fiber is configured to receive the emitted light from the at least one sensor layer.

The dressing system of the present disclosure may be placed on a skin of a user at or proximal to a monitoring site for sensing and monitoring the presence of the analyte at the monitoring site. Specifically, the dressing system may be adhered to the skin of the user at or proximal to the monitoring site by the adhesive layer.

The dressing system may be configured to sense the presence of the analyte based on one or more of an optical intensity, a photoluminescence lifetime, and a wavelength of the emitted light. The dressing system may be further configured to monitor one or more parameters related to the analyte. In some cases, the one or more parameters may include an oxygen concentration at the monitoring site. Oxygen is typically an efficient quencher of fluorescence. That is, oxygen may decrease the optical intensity of the emitted light. Therefore, a decrease in the optical intensity of the emitted light may be detected by the dressing system for sensing oxygen at a monitoring site. Therefore, the dressing system may be configured to sense the presence of oxygen. In some other cases, the one or more parameters may include a blood pressure, a temperature, a pH value, a glucose level, and an infection status. The dressing system may therefore enable in situ and real-time monitoring of the one or more parameters.

The dressing system may further assist a wound healing process by promoting tissue growth, and providing anti-bacterial and anti-inflammatory effects to the monitoring site. Specifically, the excitation light and the emitted light may promote tissue growth, and provide anti-bacterial and anti-inflammatory effects at the monitoring site.

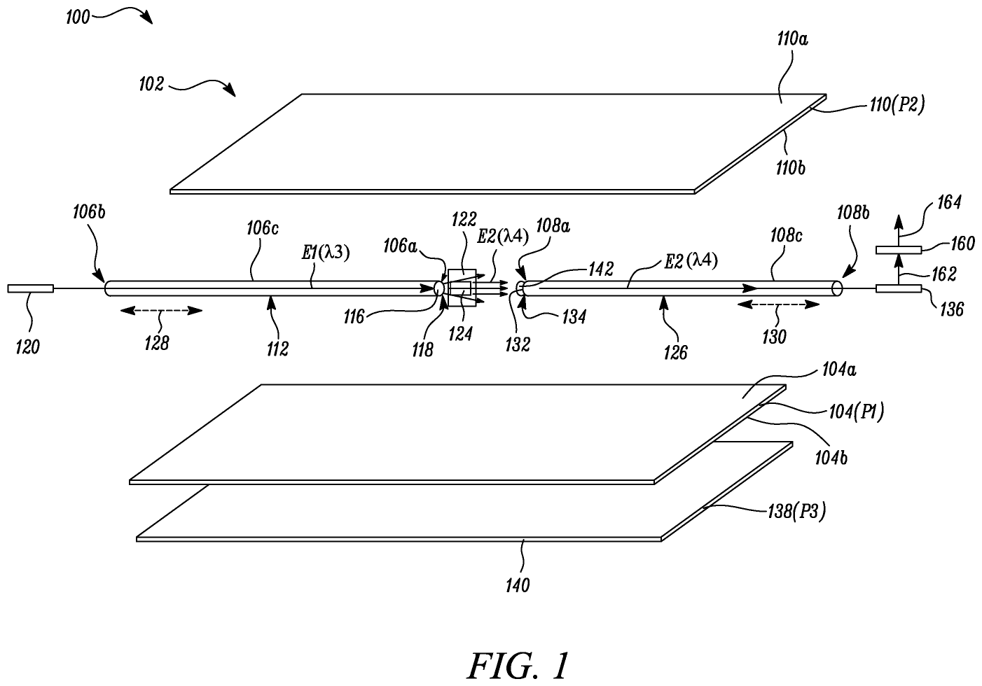

Referring now to figures, FIG. 1 illustrates an exploded perspective view of a dressing system 100 for sensing a presence of an analyte according to an embodiment of the present disclosure.

In the illustrated embodiment of FIG. 1, the dressing system 100 includes a dressing 102. The dressing 102 may be placed on a skin of a user. In some cases, the dressing 102 may be placed on the skin of the user at a monitoring site, such that the dressing 102 wholly covers the monitoring site. In some other cases, the dressing 102 may be placed proximal to the monitoring site, such that the dressing 102 at least partially covers the monitoring site. The monitoring site may include any region of the skin of the user at which sensing of the analyte may be desired, such as a wounded skin region, a healthy skin region, and so forth.

The dressing system 100 includes a first layer 104. Specifically, in the illustrated embodiment of FIG. 1, the dressing 102 includes the first layer 104. The first layer 104 includes a first major surface 104a and a second major surface 104b opposite to the first major surface 104a. The first layer 104 may include any suitable permeable material, such as polyester, cotton, rayon, polypropylene, wood pulp, polyurethanes, and the like. In some embodiments, the first layer 104 may include foam. The first layer 104 has a first permeability P1 to the analyte. In some embodiments, the first permeability P1 of the first layer 104 to the analyte may be sufficiently high to allow the analyte to pass through the first layer 104. For example, the first permeability P1 of the first layer 104 may allow the analyte from the monitoring site to pass through the first layer 104. In some embodiments, the first layer 104 may include an adhesive material disposed on the second major surface 104b. In some embodiments, the first layer 104 has the first permeability P1 to the analyte with the adhesive material disposed on the second major surface 104b. The adhesive material disposed on the second major surface 104b of the first layer 104 may allow the dressing system 100 to be placed on the skin of the user.

The dressing system 100 further includes a second layer 110. Specifically, in the illustrated embodiment of FIG. 1, the dressing 102 includes the second layer 110. In the illustrated embodiment of FIG. 1, the second layer 110 includes a first major surface 110a and a second major surface 110b opposite to the first major surface 110a.

The second layer 110 faces the first major surface 104a of the first layer 104. Specifically in the illustrated embodiment of FIG. 1, the second major surface 110b of the second layer 110 faces the first major surface 104a of the first layer 104. The second layer 110 may include any suitable permeable material, such as polyester, cotton, rayon, polypropylene, wood pulp, polyurethanes,. and the like. In some embodiments, the second layer 110 may include foam.

The second layer 110 has a second permeability P2 to the analyte less than the first permeability P1 of the first layer 104. In other words, the second permeability P2 of the second layer 110 to the analyte is less than the first permeability P1 of the first layer 104 to the analyte. In some embodiments, the first permeability P, is greater than the second permeability P2 by a factor of at least 2. In other words, in some embodiments, the first permeability P1 of the first layer 104 may be greater than two times the second permeability P2 of the second layer 110. The second permeability P2 of the second layer 110 may be low to at least partially restrict a passage of the analyte through the second layer 110. Therefore, in some cases, the first layer 104 and the second layer 110 may trap the analyte (e.g., oxygen) therebetween. This may improve sensing and monitoring of the analyte by the dressing system 100.

The dressing system 100 further includes a first fiber 112. Specifically, in the illustrated embodiment of FIG. 1, the dressing 102 includes the first fiber 112. The first fiber 112 is at least partially disposed between the first layer 104 and the second layer 110. In some embodiments, the first fiber 112 may be wholly disposed between the first layer 104 and the second layer 110. In the illustrated embodiment of FIG. 1, the first fiber 112 is substantially parallel with respect to a longitudinal axis of the dressing 102. However, the first fiber 112 may be disposed in any suitable orientation, as per desired application attributes. For example, in some embodiments, the first fiber 112 may be at least partially disposed between the first layer 104 and the second layer 110 laterally, or in a spiral configuration.

In some embodiments, the first fiber 112 may include an optoelectronics fiber. In some embodiments, the first fiber 112 may include an optical fiber. In some embodiments, the optical fiber may include a polymer. The optical fiber may include, for example, a side emitting optical fiber, a light guiding optical fiber, an optical fiber with defects in a cladding, and so forth.

In the illustrated embodiment of FIG. 1, the first fiber 112 includes a first end 106a, a second end 106b opposite to the first end 106a, and an outer surface 106c. In some embodiments, the outer surface 106c may be a cladding of the first fiber 112. In some embodiments, the first fiber 112 includes a fiber tip 116 at one end of the first fiber 112. Specifically, in the illustrated embodiment of FIG. 1, the first fiber 112 includes the fiber tip 116 at the first end 106a of the first fiber 112. The first fiber 112 is configured to deliver an excitation light E1.

In some embodiments, the first fiber 112 further includes at least one light emitting region 118 (hereinafter interchangeably referred to as “the light emitting region 118”) disposed between the first layer 104 and the second layer 110 and configured to emit the excitation light E1. In some embodiments, the light emitting region 118 of the first fiber 112 may include defects on the outer surface 106c of the first fiber 112. In the illustrated embodiment of FIG. 1, the light emitting region 118 of the first fiber 112 is the fiber tip 116.

In the illustrated embodiment of FIG. 1, the dressing system 100 further includes a light source 120 optically coupled to the first fiber 112 and configured to emit the excitation light E1. In some embodiments, the excitation light E1 emitted by the light source 120 may be unpolarized. In some other embodiments, the excitation light E1 emitted by the light source 120 may be polarized (in some cases, by a polarizer). The light source 120 may include, for example, a Light Emitting Diode (LED), a Light Amplification by Stimulated Emission of Radiation (LASER) source, and so forth. Embodiments of the present disclosure are intended to include or otherwise cover any type of the light source 120, including known, related art, and/or later developed technologies to emit the excitation light E1.

In the illustrated embodiment of FIG. 1, the light source 120 is disposed proximal to the second end 106b of the first fiber 112. Further, in the illustrated embodiment of FIG. 1, the light source 120 is disposed external to the dressing 102. However, in some other embodiments, the light source 120 may be at least partially disposed within the dressing 102. Specifically, in some embodiments, the light source 120 may be at least partially disposed between the first layer 104 and the second layer 110. In some embodiments, the light source 120 may be wholly disposed between the first layer and the second layer 110. The light source 120 may include any suitable power source for operation, such as a battery.

In the illustrated embodiment of FIG. 1, the first fiber 112 is configured to receive the excitation light E1 from the light source 120. Specifically, in the illustrated embodiment of FIG. 1, the first fiber 112 receives the excitation light E1 from the second end 106b of the first fiber 112, and emits the excitation light E1 via the light emitting region 118.

The dressing system 100 further includes at least one sensor layer 122 (hereinafter interchangeably referred to as “the sensor layer 122”). Specifically, in the illustrated embodiment of FIG. 1, the dressing 102 includes the sensor layer 122. The sensor layer 122 includes a sensor material 124 disposed between the first layer 104 and the second layer 110. Further, the sensor layer 122 is configured to receive the excitation light E1 from the first fiber 112.

As discussed above, the first fiber 112 is configured to deliver the excitation light E1. Specifically, the first fiber 112 may be configured to guide the excitation light E1 received from the light source 120 and deliver the excitation light E1 to the sensor layer 122 through the light emitting region 118. More specifically, in the illustrated embodiment of FIG. 1, the first fiber 112 is configured to deliver the excitation light E1 to the sensor layer 122 through the fiber tip 116. Moreover, the sensor layer 122 is configured to emit an emitted light E2 in response to the excitation light E1. Specifically, the sensor material 124 of the sensor layer 122 may be configured to emit the emitted light E2 upon being irradiated with the excitation light E1.

In some embodiments, the sensor material 124 includes at least one of seminaphtharhodafluor (SNARF), nano particles, glucose oxidase, glucose dehydrogenase, glucose-binding proteins, boronic acid, glucuronide derivatives, maltotriose, sugar derivatives, and a fluorescent or phosphorescent material, such as porphyrin, ruthenium compound, fluorescein or its derivates.

Enzymes, such as glucose oxidase and glucose dehydrogenase, and glucose-binding proteins may be sensitive to glucose, and thus may be used for sensing glucose level at the monitoring site.

Boronic acid, glucuronide derivatives, maltotriose, and other sugar derivatives may be sensitive to infection status, and thus may be used for sensing infection status at the monitoring site.

Porphyrins, such as platinum and/or palladium metal porphyrins, ruthenium compounds (such as Ru(dpp)3), and other fluorescent chemicals may be sensitive to oxygen, and thus may be used for sensing oxygen concentration at the monitoring site.

Fluorescein and its derivatives, SNARF, and nano particles may be sensitive to pH, and thus may be used for sensing pH level at the monitoring site.

The fluorescent material may include phosphor including solid inorganic materials consisting of a host lattice, usually intentionally doped. Phosphors may be made from a suitable host material with an activator. The host materials may include oxides, nitrides and oxynitrides, sulfides, selenides, halides or silicates of zinc, cadmium, manganese, aluminum, silicon, or various other rare-earth metals. The activators may prolong an emission time (i.e., afterglow) of the phosphor. In some embodiments, the sensor material 124 may include a photoluminescent material. In some embodiments, the photoluminescent material may include quantum dots.

In some embodiment, the sensor layer 122 may be configured to diffuse the emitted light E2. In some embodiments, the sensor layer 122 may include a suitable fluid handling architecture (not shown) to facilitate mechanical interaction between the sensor material 124 and the analyte. For example, the sensor layer 122 may include a test card, a microfluidic chip, a cuvette, a tube, an array plate, a lateral flow assay, and the like.

The emitted light E2 includes a first optical property sensitive to the presence of the analyte. In some embodiments, the first optical property is at least one of an optical intensity of the emitted light E2, a photoluminescence lifetime of the emitted light E2, and a wavelength of the emitted light E2. In some examples, the photoluminescence lifetime of the emitted light E2 may be a phosphorescence lifetime of the emitted light E2. In some embodiments, the emitted light E2 includes the first optical property having a first value in an absence of the analyte and the emitted light E2 includes the first optical property having a second value in the presence of the analyte. The first value is different from the second value. In other words, the first value may be less than or greater than, but not equal to, the second value.

In some embodiments, the excitation light E1 includes a second optical property different from the first optical property and having a third value λ3. In some embodiments, the emitted light E2 includes the second optical property having a fourth value λ4 different from the third value λ3. In the illustrated embodiment of FIG. 1, the second optical property includes a wavelength. Specifically, in the illustrated embodiment of FIG. 1, the wavelength has the third value λ3 for the excitation light E1 and the fourth value λ4 for the emitted light E2.

In some embodiments, the sensor material 124 may absorb at least a portion of the excitation light E1 having the third value λ3 of the second optical property and emit the emitted light E2 having the fourth value λ4 of the second optical property. In other words, in some embodiments, the sensor material 124 may emit the emitted light E2 having the fourth value λ4 of the second optical property upon being irradiated with the excitation light E1 having the third value λ3 of the second optical property.

The dressing system 100 further includes a second fiber 126 separate from the first fiber 112. Specifically, in the illustrated embodiment of FIG. 1, the dressing 102 includes the second fiber 126. The second fiber 126 is disposed between the first layer 104 and the second layer 110. In some embodiments, the second fiber 126 may be at least partially disposed between the first layer 104 and the second layer 110. In some other embodiments, the second fiber 126 may be wholly disposed between the first layer 104 and the second layer 110. In the illustrated embodiment of FIG. 1, the second fiber 126 is disposed substantially parallelly with respect to the longitudinal axis of the dressing 102. However, the second fiber 126 may be disposed in any suitable orientation corresponding to the first fiber 112, as per desired application attributes.

In some embodiments, the second fiber 126 may include an optoelectronics fiber. In some embodiments, the second fiber 126 may include an optical fiber. In some embodiments, the optical fiber may include a polymer. In some embodiments, the optical fiber may include a side emitting optical fiber, a light guiding optical fiber, an optical fiber with defects in a cladding, and so forth.

In the illustrated embodiment of FIG. 1, the second fiber 126 includes a first end 108a, a second end 108b opposite to the first end 108a, and an outer surface 108c. In some embodiments, the outer surface 108c may be a cladding of the second fiber 126.

In some embodiments, the second fiber 126 includes a fiber tip 132 at one end of the second fiber 126. Specifically, in the illustrated embodiment of FIG. 1, the second fiber 126 includes the fiber tip 132 at the first end 108a of the second fiber 126. The second fiber 126 is configured to receive the emitted light E2 from the sensor layer 122.

In some embodiments, the second fiber 126 includes at least one light receiving region 134 (hereinafter interchangeably referred to as “the light receiving region 134”) disposed between the first layer 104 and the second layer 110. In some embodiments, the light receiving region 134 is configured to receive the emitted light E2 from the sensor layer 122. In some embodiments, the light receiving region 134 may include defects on the outer surface 108c of the second fiber 126. In the illustrated embodiment of FIG. 1, the light receiving region 134 of the second fiber 126 is the fiber tip 132.

In some embodiments, the first fiber 112 defines a first longitudinal axis 128 along its length. In some embodiments, the second fiber 126 defines a second longitudinal axis 130. In some embodiments, the second longitudinal axis 130 may be along a length of the second fiber 126. In the illustrated embodiment of FIG. 1, the second fiber 126 is spaced apart from the first fiber 112 and aligned with the first fiber 112 along the first longitudinal axis 128. Specifically, in the illustrated embodiment of FIG. 1, the second fiber 126 is spaced apart from the first fiber 112, and the second longitudinal axis 130 of the second fiber 126 is aligned with the first longitudinal axis 128 of the first fiber 112.

In the illustrated embodiment of FIG. 1, the sensor layer 122 is disposed between the first fiber 112 and the second fiber 126. More specifically, in the illustrated embodiment of FIG. 1, the sensor layer 122 is disposed between the fiber tip 116 of the first fiber 112 and the fiber tip 132 of the second fiber 126. However, in some embodiments, the sensor layer 122 is disposed on the fiber tip 116 of the first fiber 112.

Further, in some other embodiments, the sensor layer 122 is disposed on the fiber tip 132 of the second fiber 126. The dressing system 100 may also include the sensor layer 122 in other arrangements. Specifically, in some embodiments, the sensor layer 122 is disposed on at least one of the outer surface 106c of the first fiber 112 and the outer surface 108c of the second fiber 126. Moreover, in some other embodiments, the sensor layer 122 may be added into the first fiber 112 and/or the second fiber 126 during manufacture.

In the illustrated embodiment of FIG. 1, the dressing system 100 further includes at least one optical filter 142 (hereinafter interchangeably referred to as “the optical filter 142”) configured to receive the emitted light E2 from the sensor layer 122. The optical filter 142 may selectively transmit light in a particular range of wavelengths to the second fiber 126 and absorb light outside the particular range of wavelengths. Specifically, the optical filter 142 may selectively transmit the emitted light E2 to the second fiber 126 and absorb the excitation light E1. Thus, the second fiber 126 may receive the emitted light E2 with the excitation light E1 filtered out by the optical filter 142. This may improve an accuracy of sensing and monitoring of the analyte by the dressing system 100. In some embodiments, the optical filter 142 may include an interference filter and/or a dichroic filter. In some other embodiments, the optical filter 142 may include an absorptive filter. Moreover, in yet other embodiments, the optical filter 142 may include multiple optical filters having hybrid dielectric-absorptive configurations. Such hybrid configurations may be important for pixelated light detectors.

In the illustrated embodiment of FIG. 1, the optical filter 142 is disposed between the first fiber 112 and the second fiber 126. Specifically, in the illustrated embodiment of FIG. 1, the optical filter 142 is disposed on the fiber tip 132 of the second fiber 126. In such embodiments, the optical filter 142 may be a coating disposed on the fiber tip 132 of the second fiber 126. Moreover, in some embodiments, the second fiber 126 may have a large diameter and numerical aperture (NA) to improve signal-to-noise ratio (SNR). This may further improve an accuracy of sensing and monitoring of the analyte by the dressing system 100.

In some embodiments, the optical filter 142 includes a third optical property. In some embodiments, the third optical property includes an optical transmittance of the optical filter 142. However, in some other embodiments, the third optical property may include at least one of an optical reflectance of the optical filter 142 and an optical absorption of the optical filter 142.

In some embodiments, the dressing system 100 further includes a light detector 136 optically or electrically coupled to the second fiber 126. In some embodiments, the light detector 136 is further configured to receive an optical signal or an electrical signal corresponding to the emitted light E2.

As used herein, the term “light detector” refers broadly to any device that may be configured to receive an optical signal and/or an electrical signal from a fiber (an optical fiber or an optoelectronics fiber) and may generate a signal corresponding to the optical signal and/or the electrical signal. The signal generated by the light detector may be processed by a processor.

In the illustrated embodiment of FIG. 1, the light detector 136 is optically coupled to the second fiber 126 and configured to receive an optical signal corresponding to the emitted light E2. Specifically, in the illustrated embodiment of FIG. 1, the light detector 136 is configured to receive the emitted light E2. In other words, in the illustrated embodiment of FIG. 1, the optical signal is the emitted light E2.

In the illustrated embodiment of FIG. 1, the light detector 136 is disposed proximal to the second end 108b of the second fiber 126 and along the second longitudinal axis 130. In some embodiments, the light detector 136 may be a miniaturized device disposed between the first layer 104 and the second layer 110 of the dressing system 100. In some other embodiments, the light detector 136 may be disposed external to the dressing system 100. In the illustrated embodiment of FIG. 1, the light detector 136 includes at least one of a photodetector, a camera, and a spectrometer. However, in some embodiments, the light detector 136 includes at least one of a photodetector, a camera, a spectrometer, a multimeter, and an oscilloscope.

In the illustrated embodiment of FIG. 1, the dressing system 100 further includes a processor 160 communicably coupled to the light detector 136. In some embodiments, the processor 160 may be disposed between the first layer 104 and the second layer 110 of the dressing system 100. In some other embodiments, the processor 160 may be disposed external to the dressing system 100.

In some embodiments, the processor 160 is configured to receive an input signal 162 from the light detector 136 corresponding to the emitted light E2. In some embodiments, the processor 160 is further configured to generate an output signal 164 indicative of one or more parameters based on the input signal 162. In some embodiments, the one or more parameters are at least one of an oxygen concentration, a blood pressure, a temperature, a pH value, a glucose level, and an infection status.

In the illustrated embodiment of FIG. 1, the dressing system 100 further includes an adhesive layer 138. Specifically, in the illustrated embodiment of FIG. 1, the dressing 102 includes the adhesive layer 138. In the illustrated embodiment of FIG. 1, the adhesive layer 138 is disposed on the second major surface 104b of the first layer 104. In the illustrated embodiment of FIG. 1, the adhesive layer 138 includes an adhesive material 140. In some embodiments, the adhesive material 140 may include a pressure sensitive adhesive, a heat activated adhesive (e.g., a hot melt adhesive), and the like. In some embodiments, the adhesive material 140 may include an optical clear adhesive (OCA). In some embodiments, the adhesive layer 138 may include multiple layers, and the adhesive material 140 of the multiple layers may be same or different. In some embodiments, the adhesive layer 138 may include a backing. with the adhesive material 140 disposed on both major surfaces of the backing. In some embodiments, the adhesive material 140 may be in direct contact with the skin of the user when the dressing 102 is placed on the monitoring site. In some embodiments, the adhesive layer 138 may be hypoallergenic.

In some embodiments, the adhesive material 140 may include an antimicrobial agent. The antimicrobial agent may inhibit or essentially prevent growth of microbes on the skin of the patient where the dressing system 100 may be placed. Examples of suitable antimicrobial agents include, but are not limited to, iodine, hydrogen peroxide, benzalkonium chloride, and aluminum chlorohydrate.

As discussed above, in some embodiments, the first layer 104 may include the adhesive material (e.g., the adhesive material 140) directly disposed on the second major surface 104b. Therefore, in such embodiments, the adhesive layer 138 may be omitted from the dressing system 100.

In some embodiments, the adhesive layer 138 has a third permeability P3 to the analyte greater than or equal to the first permeability P1 of the first layer 104. In some embodiments, the third permeability P3 of the adhesive layer 138 is greater than the second permeability P2 of the second layer 110. Specifically, in some embodiments, the third permeability P3 of the adhesive layer 138 is greater than the second permeability P2 by a factor of at least 1.5. In other words, in some embodiments, the third permeability P3 of the adhesive layer 138 is greater than 1.5 times of the second permeability P2 of the second layer 110. Thus, the third permeability P3 of the adhesive layer 138 may also be sufficiently high to allow the analyte to pass through the adhesive layer 138. Specifically, the third permeability P3 of the adhesive layer 138 may allow the first layer 104 and the second layer 110 to trap the analyte therebetween, thereby improving sensing and monitoring of the analyte by the dressing system 100.

The dressing system 100 may be placed on the skin of the user. Specifically, the dressing system 100 may be placed on the skin of the user at or proximal to the monitoring site for sensing and monitoring the presence of the analyte at the monitoring site. More specifically, the dressing 102 may be adhered to the skin of the user at or proximal to the monitoring site by the adhesive layer 138 or the first layer 104.

In some cases, the analyte may include oxygen. Therefore, in some embodiments, the dressing system 100 may be configured for sensing and monitoring an oxygen concentration of the skin and tissue at the monitoring site. However, in some other embodiments, the dressing system 100 may be configured to sense and monitor a blood pressure, a temperature, a pH value, a glucose level, and/or an infection status at the monitoring site.

In some cases, the dressing system 100 may further assist a wound healing process by promoting tissue growth, and providing anti-bacterial and anti-inflammatory effects to the monitoring site. Specifically, in some cases, the excitation light E1 and the emitted light E2 may promote tissue growth and provide anti-bacterial and anti-inflammatory effects at the monitoring site.

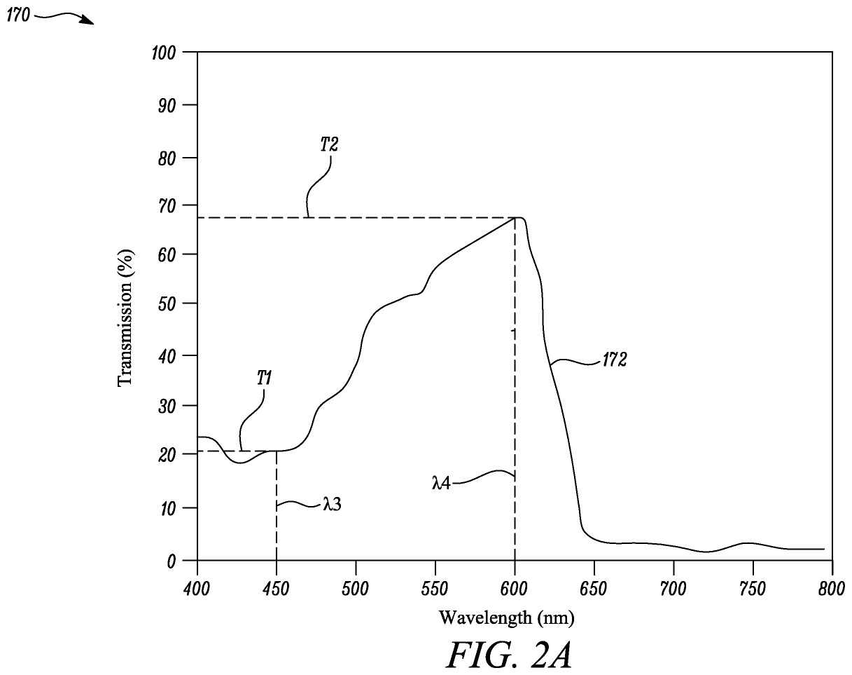

FIG. 2A illustrates a graph 170 depicting optical transmittance percentage versus wavelength of the optical filter 142 of FIG. 1 according to an embodiment of the present disclosure. Wavelength is expressed in nanometers (nm) in the abscissa (X-axis). Optical transmittance is expressed as transmission percentage in the left ordinate (left Y-axis).

Referring to FIGS. 1 and 2A, as discussed above, in some embodiments, the second optical property includes the wavelength. Specifically, in some embodiments, the wavelength has the third value λ3 for the excitation light E1 and the fourth value λ4 for the emitted light E2. Further, in some embodiments, the third optical property of the optical filter 142 includes the optical transmittance of the optical filter 142.

In the graph 170, the optical transmittance percentage of the optical filter 142 versus the wavelength of each of the excitation light E1 and the excited light E2 is depicted by an optical curve 172.

As depicted by the optical curve 172, in some embodiments, the third optical property has respective fifth and sixth values T1, T2 in response to the third and fourth values λ3, λ4 of the second optical property. Specifically, in some embodiments, the optical transmittance of the optical filter 142 has the fifth value T1 in response to the third value λ3 of the wavelength, and the optical transmittance of the optical filter 142 has the sixth value T2 in response to the fourth value λ4 of the wavelength.

In the graph 170, the third value λ3 of the wavelength is about 450 nm, and the fourth value λ4 of the wavelength is about 600 nm. In some embodiments, the sixth value T2 is greater the fifth value T1 by a factor of at least 2. In other words, in some embodiments, the sixth value T2 is greater than twice the fifth value T1. As depicted by the graph 170, in some embodiments, the fifth value T1 is about 20% in response to the third value λ3 of the wavelength and the sixth value T2 is about 67% in response to the fourth value λ4 of the wavelength. Therefore, in some embodiments, the optical filter 142 may transmit only 20% of the excitation light E1 to the second fiber 126, and transmit 67% of the emitted light E2 to the second fiber 126. Therefore, the optical filter 142 may improve the accuracy of sensing and monitoring of the analyte by the dressing system 100.

FIG. 2B illustrates a graph 175 depicting optical reflectance percentage versus wavelength of each of the first layer 104 and the second layer 110 of FIG. 1 according to an embodiment of the present disclosure. Wavelength is expressed in nanometers (nm) in the abscissa (X-axis). Optical reflectance is expressed as reflection percentage in the left ordinate (left Y-axis).

Referring to FIGS. 1 and 2B, as discussed above, in some embodiments, the wavelength has the third value λ3 for the excitation light E1 and the fourth value λ4 for the emitted light E2.

In some embodiments, the first layer 104 includes an optical reflectance having respective seventh and eighth values V7, V8 in response to the third and fourth values λ3, λ4 of the wavelength. In the graph 175, the third value λ3 of the wavelength is about 450 nm, and the fourth value λ4 of the wavelength is about 600 nm.

Further, in the graph 175, the optical reflectance versus wavelength of the first layer 104 is depicted by an optical curve 176. As depicted by the optical curve 176, the optical reflectance has the seventh value V7 in response to the third value λ3 of the wavelength, and the eighth value V8 in response to the fourth value λ4 of the wavelength.

In some embodiments, the seventh value V7 and the eighth value V8 are within 10% of each other. As depicted by the graph 175, the seventh value V7 is about 67% and the eighth value V8 is about 76%. Therefore, in some embodiments, the first layer 104 may substantially reflect the excitation light E1 and the emitted light E2.

Further, in some embodiments, the second layer 110 includes an optical reflectance having respective ninth and tenth values V9, V10 in response to the third and fourth values λ3, λ4 of the wavelength.

In the graph 175, the optical reflectance versus wavelength of the second layer 110 is depicted by an optical curve 178. As depicted by the optical curve 178, the optical reflectance has the ninth value V9 in response to the third value λ3 of the wavelength, and the tenth value V10 in response to the fourth value λ4 of the wavelength. In other words, the optical reflectance of the second layer 110 has the ninth value V9 in response to the wavelength λ3, and the tenth value V10 in response to the wavelength λ4. In some embodiments, the ninth value V9 and the tenth value V10 are within 10% of each other. As depicted by the graph 175, in some embodiments, the ninth value V9 is about 80% and the tenth value V10 is about 89%. Therefore, in some embodiments, the second layer 110 may substantially reflect the excitation light E1 and the emitted light E2.

Hence, the first layer 104 and the second layer 110 may have high optical reflectance for each of the excitation light E1 and the emitted light E2. Thus, the first layer 104 and second layer 110 may form a light recycling cavity therebetween. The light recycling cavity formed by the first layer 104 and the second layer 110 may improve sensing and monitoring of the analyte by the dressing system 100. Moreover, the dressing system 100 may not require additional reflective layers between the first layer 104 and the second layer 110 for improvement in sensing and monitoring of the analyte.

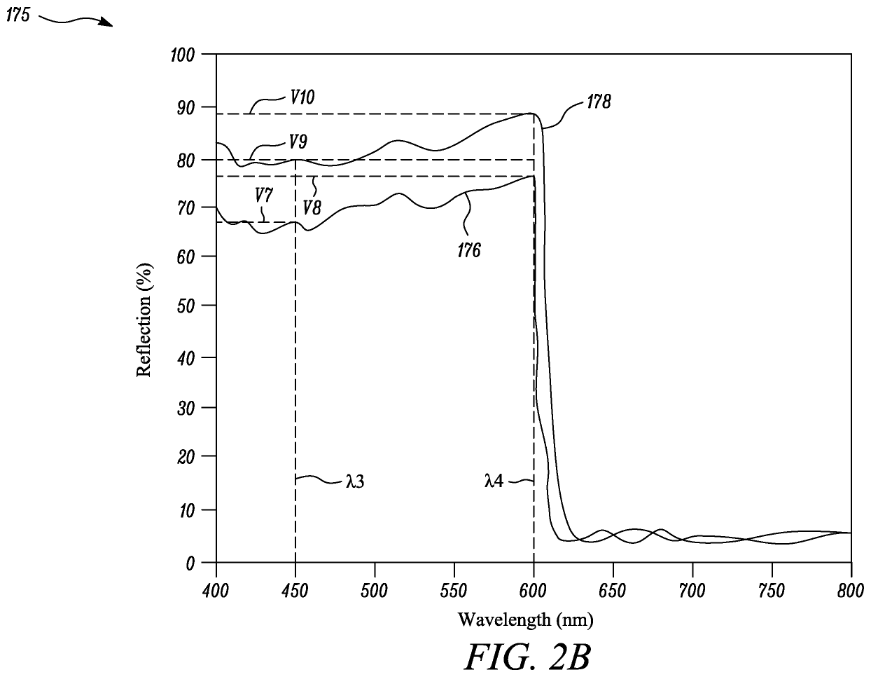

FIG. 3 illustrates a dressing system 180 for sensing a presence of an analyte according to an embodiment of the present disclosure. The dressing system 180 is similar to the dressing system 100 of FIG. 1, with like elements designated by like numbers. However, the dressing system 180 has a different configuration of the sensor layer 122 and the optical filter 142 as compared to the dressing system 100. Some elements of the dressing system 180 are not shown in FIG. 3 for illustrative purposes.

In the illustrated embodiment of FIG. 3, the sensor layer 122 is disposed between the first fiber 112 and the second fiber 126 with respect to the first longitudinal axis 128. More specifically, in the illustrated embodiment of FIG. 1, the sensor layer 122 is disposed between the fiber tip 116 of the first fiber 112 and the fiber tip 132 of the second fiber 126.

Furthermore, in the illustrated embodiment of FIG. 3, the optical filter 142 includes a plurality of optical filters 142. Specifically, in the illustrated embodiment of FIG. 3, the plurality of optical filters 142 includes a first optical filter 142a and a second optical filter 142b. However, it may be noted that the plurality of optical filters 142 may include any number of the optical filters 142, as per desired application attributes.

In some embodiments, the optical filter 142 is disposed on the fiber tip 132 of the second fiber 126. Specifically, in the illustrated embodiment of FIG. 3, the first optical filter 142a is disposed on the fiber tip 132 of the second fiber 126. Further, in some embodiments, the optical filter 142 is at least partially disposed within the second fiber 126. Specifically, in the illustrated embodiment of FIG. 3, the second optical filter 142b is at least partially disposed within the second fiber 126. In such embodiments, the second optical filter 142b may be an in-fiber coating disposed within the second fiber 126. In some other embodiments, the second optical filter 142b may include a fiber grating, a Fabry-Pérot cavity, and the like.

The plurality of optical filters 142 may further improve selective transmission of the emitted light E2 to the second fiber 126, while absorbing the excitation light E1 compared to a single optical filter 142. Thus, the second fiber 126 may receive the emitted light E2 with the excitation light E1 substantially filtered out by the plurality of optical filters 142. This may further improve an accuracy of sensing and monitoring of the analyte by the dressing system 180.

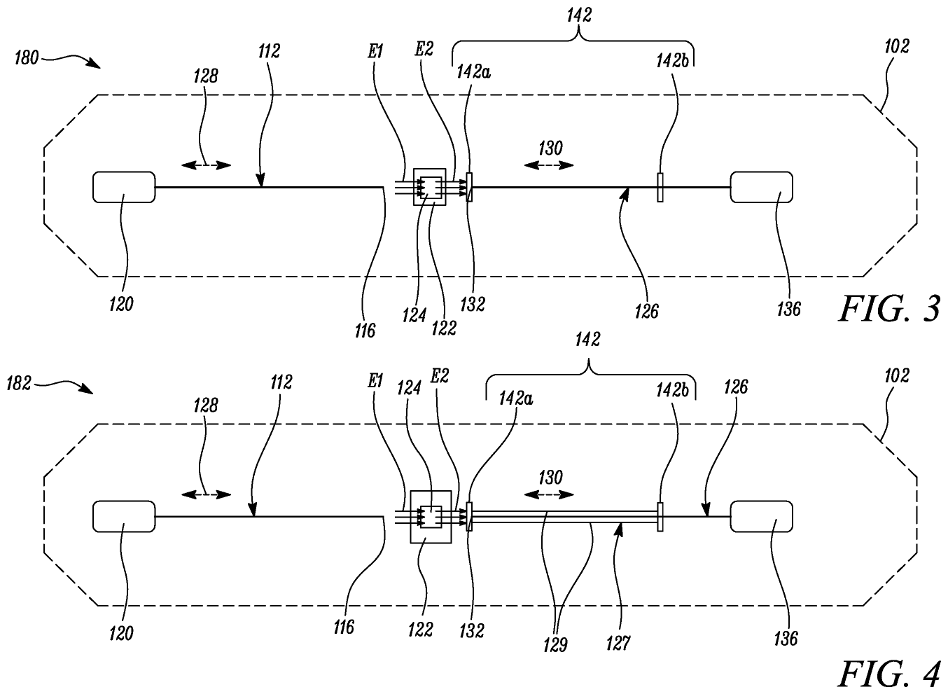

FIG. 4 illustrates a dressing system 182 for sensing a presence of an analyte according to an embodiment of the present disclosure. The dressing system 182 is similar to the dressing system 180 of FIG. 3, with like elements designated by like numbers. However, the dressing system 182 has a different configuration of the second fiber 126 as compared to the dressing system 180. Some elements of the dressing system 182 are not shown in FIG. 4 for illustrative purposes.

As shown in FIG. 4, in some embodiments, the second fiber 126 includes a fiber bundle 127 including a plurality of fibers 129 extending at least partially along a length of the second fiber 126. It may be noted that the fiber bundle 127 may include any number of the fibers 129, as per desired application attributes. For example, the fiber bundle 127 may include two, or three, or four, or five of the fibers 129. The fiber bundle 127 may include individual fibers or may be a multi-core fiber. In some embodiments, the fiber bundle 127 is disposed at least partially between the first layer 104 (shown in FIG. 1) and the second layer 110 (shown in FIG. 1). In some embodiments, each of the fibers 129 may be similar to the second fiber 126. Therefore, in some embodiments, the fiber bundle 127 may be configured to receive the emitted light E2 from the sensor layer 122. Further, in the illustrated embodiment of FIG. 4, the fiber bundle 127 is disposed between the first and second optical filters 142a, 142b. The fiber bundle 127 may improve light collection. Specifically, the fiber bundle 127 may improve collection of the emitted light E2 from the sensor layer 122. Thus, the fiber bundle 127 may improve accuracy of sensing and monitoring of the analyte by the dressing system 182.

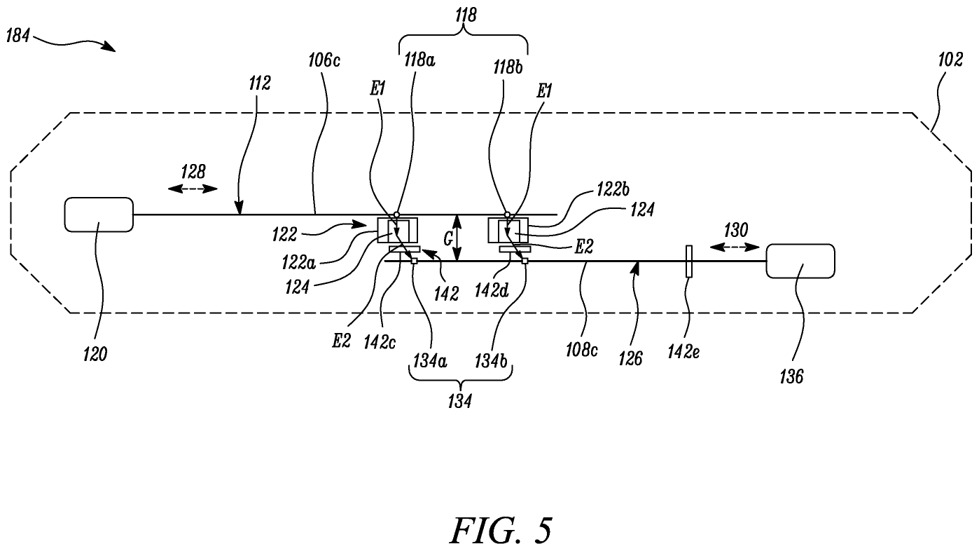

FIG. 5 illustrates a dressing system 184 for sensing a presence of an analyte according to an embodiment of the present disclosure. The dressing system 184 is similar to the dressing system 100 of FIG. 1, with like elements designated by like numbers. However, the dressing system 184 has a different configuration and arrangement of the first fiber 112 and the second fiber 126 as compared to the dressing system 100. Further, the dressing system 184 has a different configuration and arrangement of the sensor layer 122 and the optical filter 142 as compared to the dressing system 100. Some elements of the dressing system 184 are not shown in FIG. 5 for illustrative purposes.

In the illustrated embodiment of FIG. 5, the second longitudinal axis 130 is substantially parallel to and offset from the first longitudinal axis 128. Thus, in the illustrated embodiment of FIG. 5, the first fiber 112 and the second fiber 126 define a lateral gap G therebetween. In other words, in the illustrated embodiment of FIG. 5, the first longitudinal axis 128 of the first fiber 112 is substantially parallel to and laterally offset from the second longitudinal axis 130 of the second fiber 126, such that the lateral gap G is defined between the first fiber 112 and the second fiber 126.

Moreover, in the illustrated embodiment of FIG. 5, the light emitting region 118 of the first fiber 112 includes a plurality of light emitting regions 118. Each of the plurality of light emitting regions 118 is depicted by a circle in FIG. 5.

Specifically, in the illustrated embodiment of FIG. 5, the plurality of light emitting regions 118 of the first fiber 112 includes a first light emitting region 118a and a second light emitting region 118b. However, it may be noted that the plurality of light emitting regions 118 may include any number of the light emitting regions 118, as per desired application attributes. In the illustrated embodiment of FIG. 5, the first light emitting region 118a and the second light emitting region 118b include defects provided on the outer surface 106c of the first fiber 112.

Further, as shown in FIG. 5, in some embodiments, the light receiving region 134 of the second fiber 126 includes a plurality of light receiving regions 134. Each of the plurality of light receiving regions 134 is depicted by a square in FIG. 5.

Specifically, in the illustrated embodiment of FIG. 5, the plurality of light receiving regions 134 includes a first light receiving region 134a and a second light receiving region 134b. However, it may be noted that the plurality of light receiving regions 134 may include any number of the light receiving regions 134, as per desired application attributes. In the illustrated embodiment of FIG. 5, the first light receiving region 134a and the second light receiving region 134b include defects provided on the outer surface 108c of the second fiber 126.

Furthermore, in the illustrated embodiment of FIG. 5, the sensor layer 122 includes a plurality of sensor layers 122 spaced apart from each other. Specifically, in the illustrated embodiment of FIG. 5, the plurality of sensor layers 122 includes a first sensor layer 122a and a second sensor layer 122b spaced apart from the first sensor layer 122a. However, it may be noted that the plurality of sensor layers 122 may include any number of the sensor layers 122, as per desired application attributes.

In the illustrated embodiment of FIG. 5, the first sensor layer 122a is disposed between the first fiber 112 and the second fiber 126 such that the first sensor layer 122a is configured to receive the excitation light E1 from the first light emitting region 118a, and the first light receiving region 134a is configured to receive the emitted light E2 from the first sensor layer 122a. Furthermore, in the illustrated embodiment of FIG. 5, the second sensor layer 122b is disposed between the first fiber 112 and the second fiber 126 such that the second sensor layer 122b is configured to receive the excitation light E1 from the second light emitting region 118b, and the second light receiving region 134b is configured to receive the emitted light E2 from the second sensor layer 122b.

In some embodiments, the optical filter 142 includes the plurality of optical filters 142, such that each of the plurality of optical filters 142 is configured to receive the emitted light E2 from a corresponding sensor layer 122 from the plurality of sensor layers 122.

In the illustrated embodiment of FIG. 5, the plurality of optical filters 142 includes a first optical filter 142c, a second optical filter 142d, and a third optical filter 142c.

Specifically, in the illustrated embodiment of FIG. 5, the first optical filter 142c is disposed between the first fiber 112 and the second fiber 126, such that the first optical filter 142c is configured to receive the emitted light E2 from the first sensor layer 122a. In some embodiments, the first optical filter 142c may be disposed on the first light receiving region 134a of the second fiber 126.

Further, in the illustrated embodiment of FIG. 5, the second optical filter 142d is disposed between the first fiber 112 and the second fiber 126, such that the second optical filter 142d is configured to receive the emitted light E2 from the second sensor layer 122b. In some embodiments, the second optical filter 142d may be disposed on the second light receiving region 134b of the second fiber 126.

In some embodiments, the optical filter 142 is disposed between the first fiber 112 and the second fiber 126. Further, in some embodiments, the optical filter 142 is disposed proximal to the outer surface 108c of the second fiber 126. Specifically, in the illustrated embodiment of FIG. 5, each of the first optical filter 142c and second optical filter 142d is disposed between the first fiber 112 and the second fiber 126. Moreover, in the illustrated embodiment of FIG. 5, each of the first optical filter 142c and second optical filter 142d is disposed proximal to the outer surface 108c of the second fiber 126.

Further, in the illustrated embodiment of FIG. 5, the third optical filter 142e is at least partially disposed within the second fiber 126. In some embodiments, the third optical filter 142e is an in-fiber coating disposed within the second fiber 126. In some other embodiments, the third optical filter 142e may include a fiber grating, a Fabry-Pérot cavity, and the like.

The arrangement and configuration of the plurality of light emitting regions 118, the plurality of sensor layers 122, the plurality of optical filters 142, and the plurality of light receiving regions 134 of the dressing system 184 may allow the processor 160 (shown in FIG. 1) to determine an average value of the one or more parameters at the monitoring site. Therefore, the dressing system 184 may improve an accuracy of sensing and monitoring of the analyte by reducing anomalous values that may occur in single point measurements.

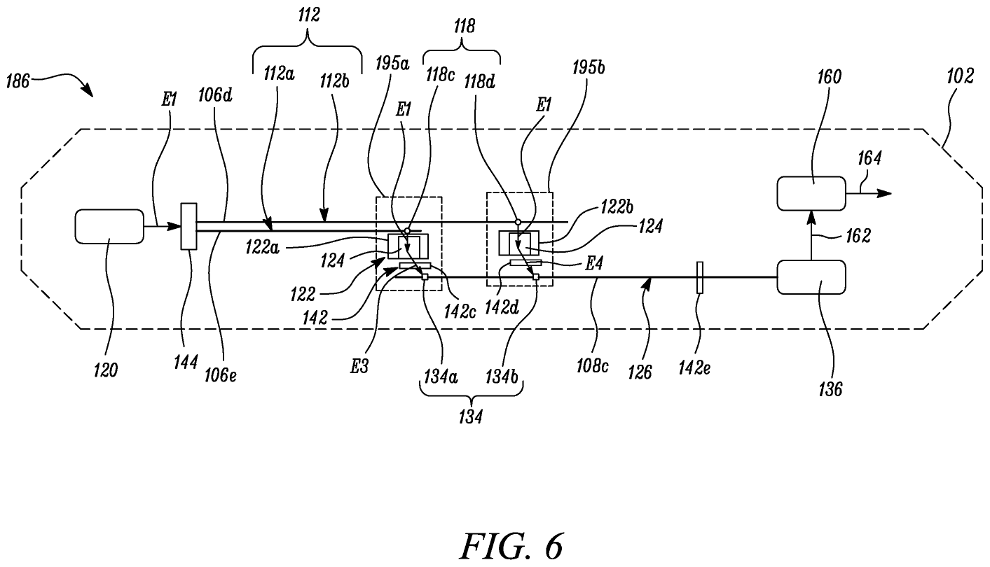

FIG. 6 illustrates a dressing system 186 for sensing a presence of an analyte according to an embodiment of the present disclosure. The dressing system 186 is similar to the dressing system 184 of FIG. 5, with like elements designated by like numbers. However, the dressing system 186 has a different configuration of the first fiber 112. Further, the first sensor layer 122a and the second sensor layer 122b have a specific arrangement with respect to the skin of the user. Moreover, the dressing system 186 includes additional elements as compared to the dressing system 184. Some elements of the dressing system 186 are not shown in FIG. 6 for illustrative purposes.

In the illustrated embodiment of FIG. 6, the first fiber 112 includes a plurality of first fibers 112. Specifically, in the illustrated embodiment of FIG. 6, the plurality of first fibers 112 includes a first fiber 112a and a first fiber 112b. However, it may be noted that the plurality of first fibers 112 may include any number of the first fibers 112, as per desired application attributes.

In the illustrated embodiment of FIG. 6, the first fiber 112a includes a first light emitting region 118c, and the first fiber 112b includes a second light emitting region 118d. Each of the plurality of light emitting regions 118 is depicted by a circle in FIG. 6. Further, in the illustrated embodiment of FIG. 6, the first light emitting region 118c includes defects provided on an outer surface 106d of the first fiber 112a, and the second light emitting region 118d includes defects provided on an outer surface 106e of the first fiber 112b.

As shown in FIG. 6, in some embodiments, the dressing system 186 includes a first optical switch 144 configured to optically couple the light source 120 to the plurality of first fibers 112. In some cases, the first optical switch 144 may be configured to uniformly distribute the excitation light E1 emitted by the light source 120 to each of the plurality of first fibers 112. In some other cases, the first optical switch 144 may be configured to selectively provide the excitation light E1 emitted by the light source 120 to one or more of the plurality of first fibers 112.

In the illustrated embodiment of FIG. 6, the first sensor layer 122a is disposed proximal to a first monitoring site 195a, and the second sensor layer 122b is disposed proximal to a second monitoring site 195b. In some cases, the first monitoring site 195a may be a wounded region of the skin and the second monitoring site 195b may be a healthy region of the skin.

In some embodiments, each of the plurality of first fibers 112 is configured to provide the excitation light E1 to the corresponding sensor layer 122 from the plurality of sensor layers 122. Specifically, in the illustrated embodiment of FIG. 6, the first fiber 112a is configured to provide the excitation light E1 to the first sensor layer 122a. Furthermore, in the illustrated embodiment of FIG. 6, the first fiber 112b is configured to provide the excitation light E1 to the second sensor layer 122b. More specifically, the first fiber 112a is configured to provide the excitation light E1 to the first sensor layer 122a via the first light emitting region 118c, and the first fiber 112b is configured to provide the excitation light E1 to the second sensor layer 122b via the second light emitting region 118d.

Further, as shown in FIG. 6, in some embodiments, the first sensor layer 122a emits an emitted light E3 in response to the excitation light E1 and the second sensor layer 122b emits an emitted light E4 in response to the excitation light E1. Further, the emitted light E3 may have a value of the third optical property different from a value of the third optical property of the emitted light E4.

Thus, the dressing system 186 may enable the processor 160 to compare the third optical property of the emitted light E3 corresponding to the first monitoring site 195a with the third optical property of the emitted light E4 corresponding to the second monitoring site 195b in order to minimize a measurement error and/or a drift in measurement of the one or more parameters related to the first monitoring site 195a, and/or the second monitoring site 195b.

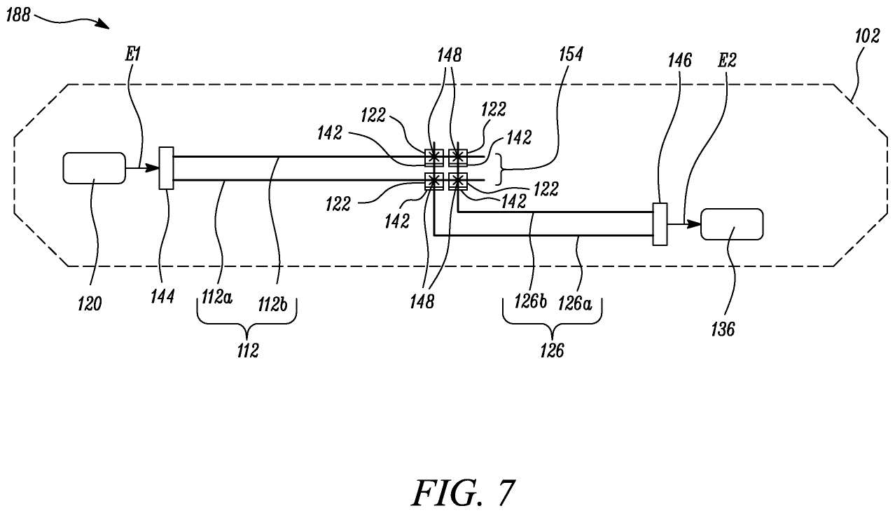

FIG. 7 illustrates a dressing system 188 for sensing a presence of an analyte according to an embodiment of the present disclosure. The dressing system 188 is similar to the dressing system 186 of FIG. 6, with like elements designated by like numbers. However, the dressing system 188 has a different configuration and arrangement of the first fiber 112 and the second fiber 126 as compared to the dressing system 186. Further, the dressing system 188 includes additional elements as compared to the dressing system 186. Some elements of the dressing system 188 are not shown in FIG. 7 for illustrative purposes.

In the illustrated embodiment of FIG. 7, the first fiber 112 includes the plurality of first fibers 112. Specifically, the plurality of first fibers 112 includes the first fiber 112a and the first fiber 112b. However, it may be noted that the plurality of first fibers 112 may include any number of the first fibers 112, as per desired application attributes.

Furthermore, in the illustrated embodiment of FIG. 7, the second fiber 126 includes a plurality of second fibers 126. In the illustrated embodiment of FIG. 7, the plurality of second fibers 126 includes a second fiber 126a and a second fiber 126b. However, it may be noted that the plurality of second fibers 126 may include any number of the second fibers 126, as per desired application attributes. In the illustrated embodiment of FIG. 7, a number of the second fibers 126 is equal to a number of the first fibers 112.

Furthermore, in the illustrated embodiment of FIG. 7, the plurality of first fibers 112 and the plurality of second fibers 126 form N×M discrete regions 148 (where N=the number of the first fibers 112 and M=the number of the second fibers 126). Each of the N×M discrete regions 148 is depicted by a cross in FIG. 7. In some embodiments, the N×M discrete regions 148 are formed at a plurality of intersections between the plurality of first fibers 112 and the plurality of second fibers 126.

Further, in the illustrated embodiment of FIG. 7, the plurality of first fibers 112 and the plurality of second fibers 126 are multiplexed to form a N×M fiber grid 154 such that the N×M fiber grid 154 monitors the N×M discrete regions 148 formed at the plurality of intersections between the plurality of first fibers 112 and the plurality of second fibers 126. In other words, in the illustrated embodiment of FIG. 7, the N×M discrete regions 148 may be discrete areas of the monitoring site that may be monitored by the dressing system 188.

In the illustrated embodiment of FIG. 7, the sensor layer 122 includes the plurality of sensor layers 122 and the optical filter 142 includes the plurality of optical filters 142. Further, in the illustrated embodiment of FIG. 7, each of the plurality of sensor layers 122 and each of the plurality of optical filters 142 are disposed in a corresponding discrete region 148 from the N×M discrete regions 148.

In some embodiments, each of the plurality of sensor layers 122 is configured to receive the excitation light E1 from a corresponding fiber 112 from the plurality of first fibers 112. In some embodiments, each of the plurality of second fibers 126 is configured to receive the emitted light E2 from the corresponding sensor layer 122 from the plurality of sensor layers 122.

In the illustrated embodiment of FIG. 7, the dressing system 182 further includes a second optical switch 146 configured to optically couple the light detector 136 to each of the plurality of second fibers 126. In some cases, the second optical switch 146 may be configured to uniformly distribute the emitted light E2 received the plurality of second fibers 126 to the light detector 136. In some other cases, the second optical switch 146 may be configured to selectively provide the emitted light E2 to the light detector 136.

The dressing system 182 may utilize the first optical switch 144, the second optical switch 146, and the N×M fiber grid 154 for sensing and monitoring of the analyte at each of the N×M discrete regions 148.

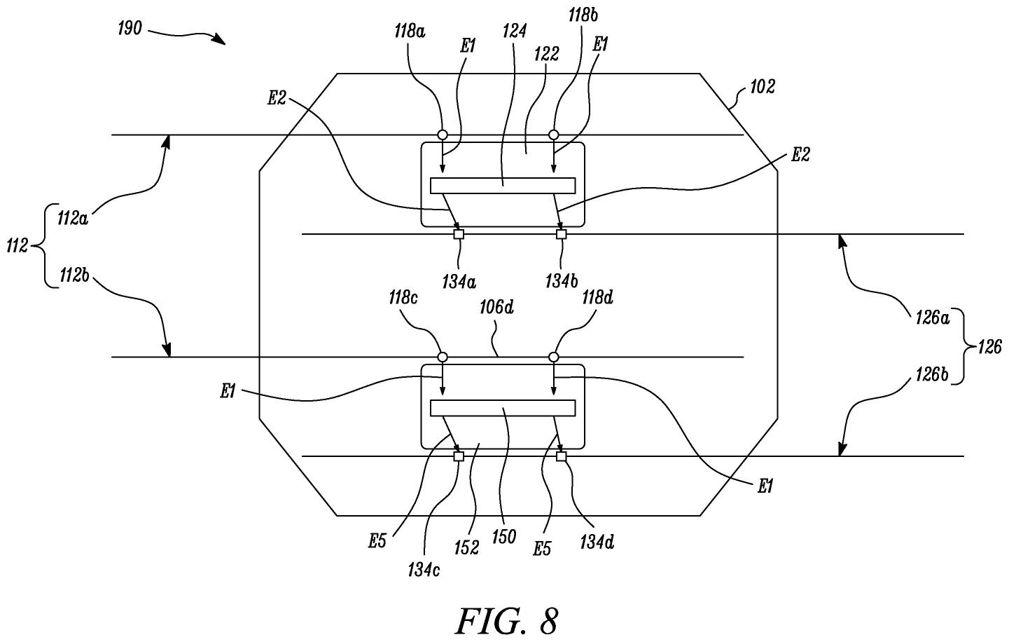

FIG. 8 illustrates a dressing system 190 for sensing a presence of an analyte according to an embodiment of the present disclosure. The dressing system 190 is similar to the dressing system 100 of FIG. 1, with like elements designated by like numbers. However, the dressing system 190 has a different configuration of the first fiber 112 and the second fiber 126 as compared to the dressing system 100. Further, the dressing system 190 has additional elements as compared to the dressing system 100. Some elements of the dressing system 190 are not shown in FIG. 8 for illustrative purposes.

In the illustrated embodiment of FIG. 8, the first fiber 112 includes the plurality of first fibers 112. Specifically, in the illustrated embodiment of FIG. 8, the plurality of first fiber 112 includes the first fiber 112a and the first fiber 112b. Further, in the illustrated embodiment of FIG. 8, the second fiber 126 includes the plurality of second fibers 126. Specifically, in the illustrated embodiment of FIG. 8, the plurality of second fibers 126 includes the second fiber 126a and the second fiber 126b.

In the illustrated embodiment of FIG. 8, the sensor layer 122 is disposed between the first fiber 112a and the second fiber 126a. In some embodiments, the sensor layer 122 emits the emitted light E2 in response to the excitation light E1 received from the first fiber 112a.

Moreover, in the illustrated embodiment of FIG. 8, the dressing system 190 further includes a reference material 150 disposed between the first layer 104 (shown in FIG. 1) and the second layer 110 (shown in FIG. 1). However, in some embodiments, the reference material 150 may be disposed on the outer surface 106d of the first fiber 112b. In the illustrated embodiment of FIG. 8, a layer 152 includes the reference material 150.

In some embodiments, the reference material 150 is configured to receive the excitation light E1 from the first fiber 112b and emit a reference emitted light E5 in response to the excitation light E1. In some embodiments, the reference material 150 is insensitive to the analyte. In other words, in some embodiments, the reference material 150 emits the reference emitted light E5 in response to the excitation light E1 such that an optical property of the reference emitted light E5 is substantially unchanged in the presence of the analyte. In some embodiment, the optical property of the reference emitted light E5 is at least one of an optical intensity of the reference emitted light E5, a photoluminescence lifetime of the reference emitted light E5, and a wavelength of the reference emitted light E5. Specifically, in some embodiments, the optical intensity of the reference emitted light E5, the photoluminescence lifetime of the reference emitted light E5, and the wavelength of the reference emitted light E5 may be substantially similar to the optical intensity of the excitation light E1, the photoluminescence lifetime of the excitation light E1, and the wavelength of the excitation light E1, respectively.

In some embodiments, the dressing system 190 may be used to calibrate the light detector 136 (shown in FIG. 1) and reduce signal fluctuation (e.g., fluctuation of the input signal 162 shown in FIG. 1) during detection of the optical intensity of the emitted light E2. Specifically, in some embodiments, the reference material 150 may be used along with the sensor material 124 of the sensor layer 122 to calibrate the light detector 136 and reduce the signal fluctuation.

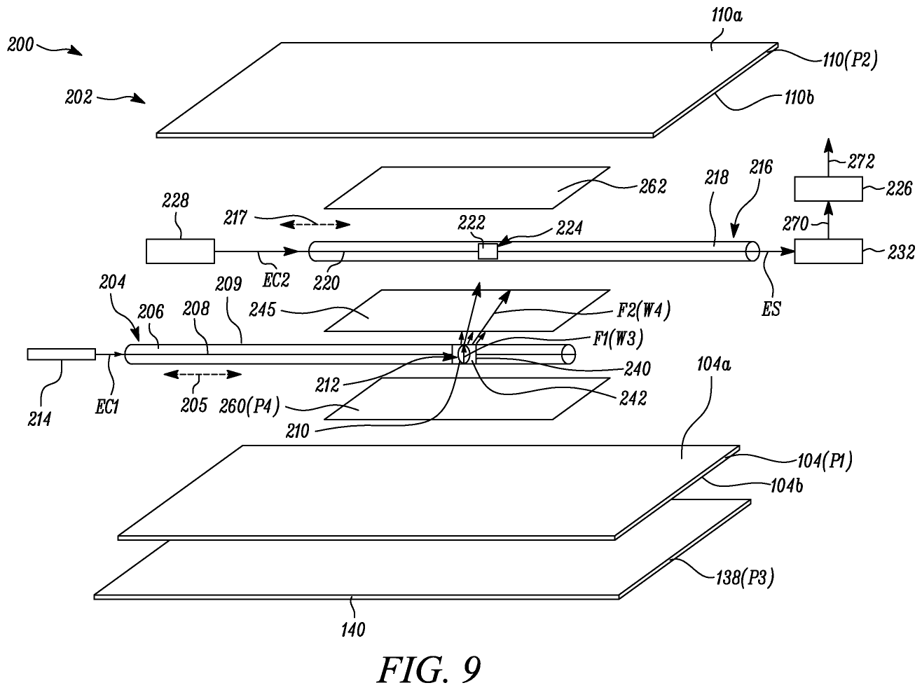

FIG. 9 illustrates an exploded view of a dressing system 200 for sensing a presence of an analyte according to an embodiment of the present disclosure. In the illustrated embodiment of FIG. 9, the dressing system 200 includes a dressing 202 configured to be placed on the skin of the user.

The dressing system 200 includes the first layer 104 including the first major surface 104a and the second major surface 104b. Specifically, in the illustrated embodiment of FIG. 9, the dressing 202 includes the first layer 104.

The dressing system 200 further includes the second layer 110. Specifically, in the illustrated embodiment of FIG. 9, the dressing 202 includes the second layer 110. In the illustrated embodiment of FIG. 9, the second layer 110 includes the first major surface 110a and the second major surface 110b. As discussed above, the second layer 110 faces the first major surface 104a of the first layer 104. Specifically, in the illustrated embodiment of FIG. 9, the second major surface 110b of the second layer 110 faces the first major surface 104a of the first layer 104.

In the illustrated embodiment of FIG. 9, the dressing system 200 further includes the adhesive layer 138. Specifically, in the illustrated embodiment of FIG. 9, the dressing 202 includes the adhesive layer 138. In the illustrated embodiment of FIG. 9, the adhesive layer 138 is disposed on the second major surface 104b of the first layer 104.

The dressing system 200 further includes a first fiber 204. Specifically, in the illustrated embodiment of FIG. 9, the dressing 202 includes the first fiber 204. The first fiber 204 is at least partially disposed between the first layer 104 and the second layer 110. In some embodiments, the first fiber 204 may be wholly disposed between the first layer 104 and the second layer 110. In the illustrated embodiment of FIG. 9, the first fiber 204 is substantially parallel with respect to a longitudinal axis of the dressing 202. However, the first fiber 204 may be disposed in any suitable orientation, as per desired application attributes. For example, in some embodiments, the first fiber 204 may be at least partially disposed between the first layer 104 and the second layer 110 laterally, or in a spiral configuration.

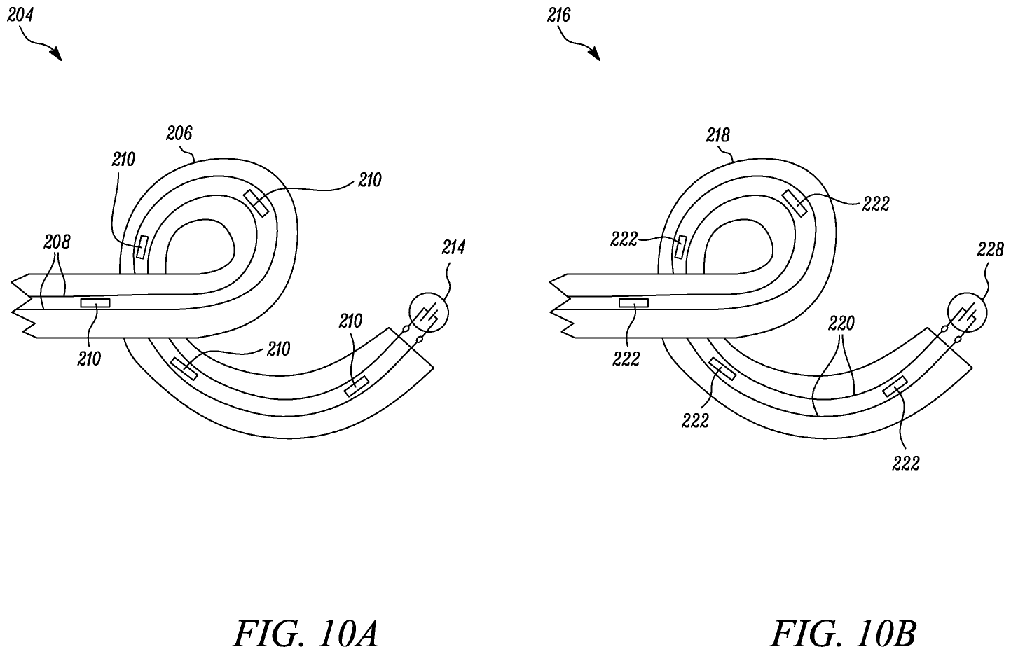

FIG. 10A illustrates the first fiber 204 of the dressing system 200 of FIG. 9 according to an embodiment of the present disclosure.

Referring to FIGS. 9 and 10A, in some embodiments, the first fiber 204 includes a fiber body 206. In some embodiments, the first fiber 204 may be fabricated from a macroscopic fiber preform thermally drawn using a suitable thermal drawing process. The fiber body 206 may include a material including, but not limited to, a thermoplastic polymer, a glass, an elastomer, a thermoset, or any other material that can flow during thermal drawing process. In some cases, conventional fiber cladding materials may be employed as the material of the fiber body 206. Examples of the conventional fiber cladding materials include Polycarbonate (PC), Poly-ethylene (PE), Cyclic Olefin copolymers (COC), Poly-methyl methacrylate (PMMA) or any other acrylic, Polysulfone (PSU), Polyetherimide (PEI), Polystyrene (PS), Polyethylene (PE), Poly-ether ether ketone (PEEK), poly-ether sulfone (PES), and the like. In some cases, semicrystalline polymers, e.g., branched PTFE or PE, may be employed as the material for the fiber body 206. In some embodiments, the fiber body 206 may be flexible.

In some embodiments, the first fiber 204 further includes at least one electrical conductor 208 (hereinafter interchangeably referred to as “the electrical conductor 208”) disposed within the fiber body 206 and extending at least partially along a length of the fiber body 206. In the illustrated embodiment of FIG. 10A, the electrical conductor 208 includes a plurality of electrical conductors 208.

In some embodiments, the electrical conductor 208 may include a material that co-flows with the material of the fiber body 206 at a common fiber draw temperature. However, the electrical conductor 208 may include other materials that do not flow at the fiber draw temperature. In either case, the electrical conductor 208 may be electrically conductive connection media. For materials that do co-flow with the material of the fiber body 206, the electrical conductor 208 may be formed of a material or materials that melt at the fiber draw temperature. In such cases, low melting-temperature metals such as Bi—Sn alloys, In-based alloys, Sn—Pb alloys, or any other suitable conducting materials that are liquid at a selected fiber draw temperature may be employed.

In some embodiments, the first fiber 204 further includes at least one light emitting device 210 (hereinafter interchangeably referred to as “the light emitting device 210”) electrically connected to the electrical conductor 208 and configured to emit an excitation light F1 in response to an electrical current EC1. In other words, the first fiber 204 is configured to deliver the excitation light F1. In some embodiments, the light emitting device 210 may include a light emitting diode (LED). Specifically, in some embodiments, the LED may emit the excitation light F1 in response to the electrical current EC1.

In the illustrated embodiment of FIG. 9A, the light emitting device 210 includes a plurality of light emitting devices 210. In some embodiments, the light emitting device 210 may include one or more conducting pads (not shown) to electrically connect the electrical conductor 208 with the light emitting device 210. In some embodiments, the light emitting device 210 may be electrically connected to the plurality of electrical conductors 208 in a parallel electrical connection. The parallel electrical connection may improve a reliability of the dressing system 200 during use, particularly when the first fiber 204 includes the plurality of light emitting devices 210.

In some embodiments, the dressing system 200 further includes a current source 214 electrically coupled to the electrical conductor 208 and configured to supply the electrical current EC1 to the light emitting device 210 via the electrical conductor 208. In other words, in some embodiments, the current source 214 is configured to supply the electrical current EC1 to the light emitting device 210 via the electrical conductor 208, such that the light emitting device 210 emits the excitation light F1. In some embodiments, the electrical conductor 208 may extend further than the fiber body 206 at one end of the first fiber 204 to electrically couple the electrical conductor 208 to the current source 214.

The current source 214 may include any device or circuitry capable of supplying the electrical current EC1 to the light emitting device 210 via the at electrical conductor 208. In some embodiments, the current source 214 is at least one of a battery, a capacitor, a wearable nanogenerator, and a wireless charging circuit. Embodiments of the present disclosure are intended to include or otherwise cover any type of the current source 214, including known, related art, and later developed technologies for supplying the electrical current EC1.

In some embodiments, the first fiber 204 further includes at least one light emitting region 212 (hereinafter interchangeably referred to as “the light emitting region 212”) disposed between the first layer 104 and the second layer 110 and configured to emit the excitation light F1. In the illustrated embodiment of FIG. 9, the light emitting region 212 is proximal to the light emitting device 210.

The dressing system 200 further includes at least one sensor layer 240 (hereinafter interchangeably referred to as “the sensor layer 240”) including a sensor material 242 disposed between the first layer 104 and the second layer 110. The sensor layer 240 and the sensor material 242 may be substantially similar to the sensor layer 122 and the sensor material 124, respectively, of FIG. 1. The sensor layer 240 is configured to receive the excitation light F1 from the first fiber 204 and emit an emitted light F2 in response to the excitation light F1.

In the illustrated embodiment of FIG. 9, the sensor layer 240 is disposed on an outer surface 209 of the first fiber 204 and proximal to the light emitting region 212. Specifically, in the illustrated embodiment of FIG. 9, the light emitting region 212 delivers the excitation light F1 emitted from the light emitting device 210 to the sensor layer 240.

The emitted light F2 includes a first optical property sensitive to the presence of the analyte. In some embodiments, the first optical property is at least one of an optical intensity of the emitted light F2, a photoluminescence lifetime of the emitted light F2, and a wavelength of the emitted light F2. In some embodiments, the emitted light F2 includes the first optical property having a first value in an absence of the analyte and the emitted light F2 includes the first optical property having a second value in the presence of the analyte. The first value is different from the second value. In other words, the first value may be less than or greater than, but not equal to, the second value.

In some embodiments, the excitation light F1 includes a second optical property different from the first optical property and having a third value W3. In some embodiments, the emitted light F2 includes the second optical property having the fourth value W4 different from the third value W3. In the illustrated embodiment of FIG. 9, the second optical property includes a wavelength. Specifically, in the illustrated embodiment of FIG. 9, the wavelength has the third value W3 for the excitation light F1 and the fourth value W4 for the emitted light F2.

The dressing system 200 further includes a second fiber 216 separate from the first fiber 204. Specifically, in the illustrated embodiment of FIG. 9, the dressing 202 includes the second fiber 216. The second fiber 216 is disposed between the first layer 104 and the second layer 110. In some embodiments, the second fiber 216 may be at least partially disposed between the first layer 104 and the second layer 110. In some embodiments, the second fiber 216 may be wholly disposed between the first layer 104 and the second layer 110. In the illustrated embodiment of FIG. 9, the second fiber 216 is disposed substantially parallelly with respect to the longitudinal axis of the dressing 202. However, the second fiber 216 may be disposed in any suitable orientation corresponding to the first fiber 204, as per desired application attributes.

FIG. 10B illustrates the second fiber 216 of the dressing system 200 of FIG. 9 according to an embodiment of the present disclosure.

Referring to FIGS. 9 and 10B, in some embodiments, the second fiber 216 includes a fiber body 218. In some embodiments, the second fiber 216 may be fabricated from a macroscopic fiber preform thermally drawn using a suitable thermal drawing process. The fiber body 218 may include a material including, but not limited to, a thermoplastic polymer, a glass, an elastomer, a thermoset, or any other material that can flow during thermal drawing process. In some cases, conventional fiber cladding materials may be employed as the material of the fiber body 218. Examples of the conventional fiber cladding materials include Polycarbonate (PC), Poly-ethylene (PE), Cyclic Olefin copolymers (COC), Poly-methyl methacrylate (PMMA) or any other acrylic, Polysulfone (PSU), Polyetherimide (PEI), Polystyrene (PS), Polyethylene (PE), Poly-ether ether ketone (PEEK), poly-ether sulfone (PES), and the like. In some cases, semicrystalline polymers, e.g., branched PTFE or PE, may be employed as the material for the fiber body 218. In some embodiments, the fiber body 218 may be flexible.

In some embodiments, the second fiber 216 includes at least one electrical conductor 220 (hereinafter interchangeably referred to as “the electrical conductor 220”) disposed within the fiber body 218 and extending at least partially along a length of the fiber body 218. In the illustrated embodiment of FIG. 10B, the electrical conductor 220 includes a plurality of electrical conductors 220.

In some embodiments, the electrical conductor 220 may include a material that co-flows with the material of the fiber body 218 at a common fiber draw temperature. However, the electrical conductor 220 may include other materials that do not flow at the fiber draw temperature. In either case, the electrical conductors 220 may be electrically conductive connection media. For materials that do co-flow with the material of the fiber body 218, the electrical conductors 220 may be formed of a material or materials that melt at the fiber draw temperature. In such cases, low melting-temperature metals such as Bi—Sn alloys, In-based alloys, Sn—Pb alloys, or any other suitable conducting materials that are liquid at a selected fiber draw temperature may be employed.

In some embodiments, the second fiber 216 further includes at least one at least one light detecting device 222 (hereinafter interchangeably referred to as “the light detecting device 222”) electrically connected to the electrical conductor 220 and configured to generate an electrical signal ES upon receiving the emitted light F1 from the sensor layer 240. In other words, the second fiber 216 is configured to receive the emitted light F2 from the sensor layer 240. The light detecting device 222 may include, for example, a photovoltaic cell, a phototransistor, a photoresistor, a phototube, a photomultiplier tube, a charge coupled device, and the like. In some embodiments, the light detecting device 222 may include a photodiode. Specifically, in some embodiments, the photodiode may convert the emitted light F2 received from the sensor layer 240 into the electrical signal ES.

In some embodiments, the light detecting device 222 may include one or more conducting pads (not shown) to electrically connect the electrical conductor 220 with the light detecting device 222. In some embodiments, the light detecting device 222 may be electrically connected to the plurality of electrical conductors 220 in a parallel electrical connection.

In some embodiments, the dressing system 200 further includes a current source 228 electrically coupled to the electrical conductor 220 of the second fiber 216 and configured to supply an electrical current EC2 to the light detecting device 222 via the electrical conductor 220. In other words, in some embodiments, the current source 228 is configured to supply the electrical current EC2 to the light detecting device 222 via the electrical conductor 220, such that the light detecting device 222 detects the emitted light F2.

In some embodiments, the current source 228 may be used to reverse bias the photodiode (e.g., PIN photodiodes, avalanche photodiodes, and the like). However, in some embodiments, the photodiode (e.g., PN photodiodes) may not require reverse biasing, and thus the current source 228 may be omitted from the dressing system 200.

The current source 228 may include any device or circuitry capable of supplying the electrical current EC2 to the light detecting device 222 via the at electrical conductor 220. In some embodiments, the current source 228 is at least one of a battery, a capacitor, a wearable nanogenerator, and a wireless charging circuit. Embodiments of the present disclosure are intended to include or otherwise cover any type of the current source 228, including known, related, and later developed technologies for supplying the electrical current EC2 to the light detecting device 222.

In the illustrated embodiment of FIG. 9, the second fiber 216 further includes at least one light receiving region 224 (hereinafter interchangeably referred to as “the light receiving region 224”). In the illustrated embodiment of FIG. 9, the light receiving region 224 is disposed between the first layer 104 and the second layer 110. In some embodiments, the light receiving region 224 is configured to receive the emitted light F2 from the sensor layer 240. In the illustrated embodiment of FIG. 9, the light receiving region 224 is proximal to the light detecting device 222.

In some embodiments, the first fiber 204 defines a first longitudinal axis 205 along its length. In some embodiments, the second fiber 216 defines a second longitudinal axis 217 that is substantially parallel and offset from the first longitudinal axis 205.

In the illustrated embodiment of FIG. 9, the dressing system 200 further includes at least one optical filter 245 (hereinafter interchangeably referred to as “the optical filter 245”). Specifically, in the illustrated embodiment of FIG. 9, the dressing 202 includes the optical filter 245. In some embodiments, the optical filter 245 is configured to receive the emitted light F2 from the sensor layer 240. The optical filter 245 may selectively transmit light in a particular range of wavelengths to the second fiber 216 and absorb light outside the particular range of wavelengths. Specifically, the optical filter 245 may selectively transmit the emitted light F2 to the second fiber 216 and absorb the excitation light F2. Thus, the second fiber 216 may receive the emitted light F2 with the excitation light F1 filtered out by the optical filter 245. This may improve an accuracy of sensing and monitoring of the analyte by the dressing system 200.

In the illustrated embodiment of FIG. 9, the optical filter 245 is disposed between the first fiber 204 and the second fiber 216. However, in some other embodiments, the optical filter 245 may be disposed on the second fiber 216.

In the illustrated embodiments of FIG. 9, the dressing system 200 further includes at least one light detector 232 (hereinafter interchangeably referred to as “the light detector 232”) electrically coupled to the second fiber 216. Further, in the illustrated embodiment of FIG. 9, the light detector is configured to receive the electrical signal ES corresponding to the emitted light F2. In some embodiments, the light detector 232 is configured to generate an input signal 270 corresponding to the electrical signal ES. The light detector 232 may include, for example, an ammeter, a multimeter, an oscilloscope, and the like. Embodiments of the present disclosure are intended to include or otherwise cover any type of the light detector 232, including known, related art, and/or later developed technologies to generate the input signal 270 in response to the electrical signal ES.

In the illustrated embodiment of FIG. 9, the dressing system 200 further includes a processor 226 communicably coupled to the light detector 232 and configured to receive the input signal 270 from the light detector 232 corresponding to the emitted light F2. As discussed above, in some embodiments, the input signal 270 corresponds to the electrical signal ES. In some embodiments, the processor 226 is further configured to generate an output signal 272 indicative of one or more parameters based on the input signal 270. In some embodiments, the one or more parameters are at least one of an oxygen concentration, a blood pressure, a temperature, a pH value, a glucose level, and an infection status.

In the illustrated embodiment of FIG. 9, the dressing system 200 further includes a first reflective layer 260. Specifically, in the illustrated embodiment of FIG. 9, the dressing 202 includes the first reflective layer 260. In the illustrated embodiment of FIG. 9, the first reflective layer 260 is disposed between the first major surface 104a of the first layer 104 and the sensor layer 240. Specifically, in the illustrated embodiment of FIG. 9, the first reflective layer 260 is disposed between the first major surface 104a of the first layer 104 and the first fiber 204.

The first reflective layer 260 may include a permeable film including any suitable type of coating that reflects the excitation light F1 and the emitted light F2. For example, the first reflective layer 260 may include a multilayer inorganic dielectric coating, a polymeric multilayer optical film (MOF) coating, a metal coating, and the like. An optical reflectance of the first reflective layer 260 may be high, such that the first reflective layer 260 reflects a substantial portion the excitation light F1 and the emitted light F2.

In some embodiments, the first reflective layer 260 has a fourth permeability P4 to the analyte. In some embodiments, the fourth permeability P4 of the first reflective layer 260 is greater than the second permeability P2 of the second layer 110.

In the illustrated embodiment of FIG. 9, the dressing system 200 further includes a second reflective layer 262. Specifically, in the illustrated embodiment of FIG. 9, the dressing 202 includes the second reflective layer 262. In the illustrated embodiment of FIG. 9, the second reflective layer 262 is disposed between the sensor layer 240 and the second layer 110. Specifically, in the illustrated embodiment of FIG. 9, the second reflective layer 262 is disposed between the sensor layer 122 and the second major surface 110b of the second layer 110. The second reflective layer 262 may include an impermeable film including any suitable type of coating that reflects the excitation light F1 and the emitted light F2. For example, the second reflective layer 262 may include a multilayer inorganic dielectric coating, a polymeric multilayer optical film (MOF) coating, a metal coating, and the like. An optical reflectance of the second reflective layer 262 may be high, such that the second reflective layer 262 reflects a substantial portion of the excitation light F1 and the emitted light F2.

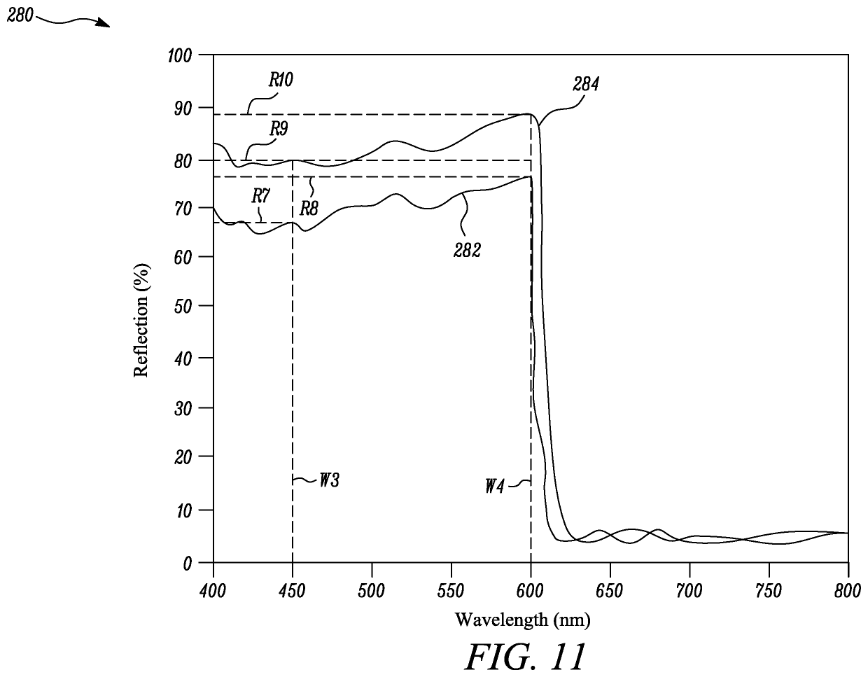

FIG. 11 illustrates a graph 280 depicting optical reflectance percentage versus wavelength of each of the first reflective layer 260 and the second reflective layer 262 of the dressing system 200 of FIG. 9 according to an embodiment of the present disclosure. Wavelength is expressed in nanometers (nm) in the abscissa (X-axis). Optical reflectance is expressed as reflection percentage in the left ordinate (left Y-axis).

Referring to FIGS. 9 and 11, as discussed above, in some embodiments, the second optical property includes the wavelength. Specifically, in some embodiments, the wavelength has the third value W3 for the excitation light F1 and the fourth value W4 for the emitted light F2.

In some embodiments, the first reflective layer 260 includes the optical reflectance having respective seventh and eighth values R7, R8 in response to the third and fourth values W3, W4 of the wavelength. Moreover, in some embodiments, the second reflective layer 262 includes the optical reflectance having respective ninth and tenth values R9, R10 in response to the third and fourth values W3, W4 of the wavelength.

In the graph 280, the optical reflectance versus wavelength of the first reflective layer 260 is depicted by an optical curve 282. As depicted by the optical curve 282, the optical reflectance has the seventh value R7 in response to the third value W3 of the wavelength, and the eighth value R8 in response to the fourth value W4 of the wavelength. In some embodiments, the seventh value R7 and the eighth value R8 are within 10% of each other. As depicted by the graph 280, in some embodiments, the seventh value R7 is about 67% and the eighth value R8 is about 76%. Therefore, in some embodiments, the first reflective layer 260 may substantially reflect the excitation light F1 and the emitted light F2.

In some embodiments, the second reflective layer includes the optical reflectance having respective ninth and tenth values R9, R10 in response to the third and fourth values W3, W4 of the wavelength.

In the graph 280, the optical reflectance versus wavelength of the second reflective layer 262 is depicted by an optical curve 284. As depicted by the optical curve 284, the optical reflectance has the ninth value R9 in response to the third value W3 of the wavelength, and the tenth value R10 in response to the fourth value W4 of the wavelength. In some embodiments, the ninth value W9 and the tenth value W10 are within 10% of each other. As depicted by the graph 280, in some embodiments, the ninth value R9 is about 80% and the tenth value R10 is about 89%. Therefore, in some embodiments, the second reflective layer 262 may substantially reflect the excitation light F1 and the emitted light F2.

Hence, the first reflective layer 260 and the second reflective layer 262 may have high optical reflectance for each of the excitation light F1 and the emitted light F2. Thus, the first reflective layer 260 and the second reflective layer 262 may form a light recycling cavity therebetween. The light recycling cavity formed by the first reflective layer 260 and the second reflective layer 262 may improve sensing and monitoring of the analyte.

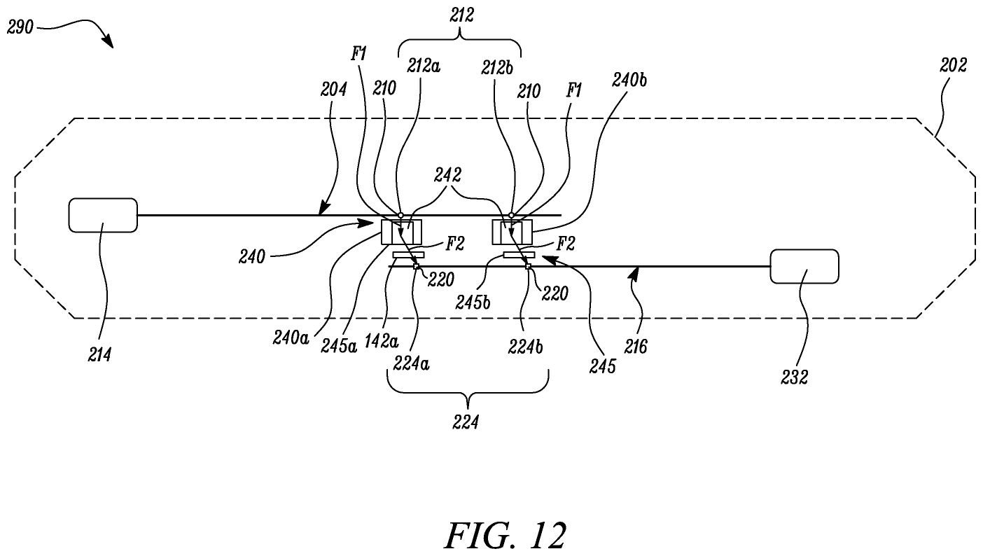

FIG. 12 illustrates a dressing system 290 for sensing a presence of an analyte according to an embodiment of the present disclosure. The dressing system 290 is similar to the dressing system 200 of FIG. 9, with like elements designated by like numbers. However, dressing system 290 has a different configuration and arrangement of the first fiber 204 and the second fiber 216 as compared to the dressing system 200. Further, the dressing system 290 has a different arrangement of the sensor layer 240 and the optical filter 245 as compared to the dressing system 200. Some elements of the dressing system 290 are not shown in FIG. 12 for illustrative purposes.

In the illustrated embodiment of FIG. 12, the light emitting region 212 of the first fiber 204 includes a plurality of light emitting regions 212. Each of the plurality of light emitting regions 212 is depicted by a circle in FIG. 12.

Specifically, in the illustrated embodiment of FIG. 12, the plurality of light emitting regions 212 includes a first light emitting region 212a and a second light emitting region 212b. However, it may be noted that the plurality of light emitting regions 212 may include any number of the light emitting regions 212, as per desired application attributes. In the illustrated embodiment of FIG. 12, the first light emitting region 212a and the second light emitting region 212b include respective light emitting devices 210.

In the illustrated embodiment of FIG. 12, the light receiving region 224 of the second fiber 216 includes a plurality of light receiving regions 224. Each of the plurality of light receiving regions 212 is depicted by a square in FIG. 12.

Specifically, in the illustrated embodiment of FIG. 12, the plurality of light receiving regions 224 includes a first light receiving region 224a and a second light receiving region 224b. However, it may be noted that the plurality of light receiving regions 224 may include any number of the light receiving regions 224, as per desired application attributes. In the illustrated embodiment of FIG. 12, the first light receiving region 224a and the second light receiving region 224b include respective light detecting devices 222.

Further, in the illustrated embodiment of FIG. 12, the sensor layer 240 includes a plurality of sensor layers 240 spaced apart from each other. Specifically, in the illustrated embodiment of FIG. 12, the plurality of sensor layers 240 include a first sensor layer 240a and a second sensor layer 240b spaced apart from the first sensor layer 240a. However, in some other embodiments, the plurality of sensor layers 240 may include any number of the sensor layers 240, as per desired application attributes.

In the illustrated embodiment of FIG. 12, the first sensor layer 240a is disposed between the first fiber 204 and the second fiber 216 such that the first sensor layer 240a is configured to receive the excitation light F1 from the first light emitting region 212a, and the first light receiving region 224a is configured to receive the emitted light F2 from the first sensor layer 240a. Furthermore, in the illustrated embodiment of FIG. 12, the second sensor layer 240b is disposed between the first fiber 204 and the second fiber 216 such that the second sensor layer 240b is configured to receive the excitation light F1 from the second light emitting region 212b, and the second light receiving region 224b is configured to receive the emitted light F2 from the second sensor layer 240b.

In the illustrated embodiment of FIG. 12, the optical filter 245 includes a plurality of optical filters 245. Specifically, in the illustrated embodiment of FIG. 12, the plurality of optical filters 245 includes a first optical filter 245a and a second optical filter 245b. In the illustrated embodiment of FIG. 12, the first optical filter 245a is disposed between the first fiber 204 and the second fiber 216 proximal to the first light emitting region 212a and the first light receiving region 224a. Moreover, in the illustrated embodiment of FIG. 12, the second optical filter 245b is disposed between the first fiber 204 and the second fiber 216 proximal to the second light emitting region 212b and the second light receiving region 224b.

In some embodiments, the optical filter 245 includes the plurality of optical filters 245, such that each of the plurality of optical filters 245 is configured to receive the emitted light F2 from a corresponding sensor layer 240 from the plurality of sensor layers 240.

Specifically, in the illustrated embodiment of FIG. 12, the first optical filter 245a is disposed between the first fiber 112 and the second fiber 216, such that the first optical filter 245a is configured to receive the emitted light F2 from the first sensor layer 240a from the plurality of sensor layers 240.

Specifically, in the illustrated embodiment of FIG. 12, the second optical filter 245b is disposed between the first fiber 112 and the second fiber 216, such that the second optical filter 245b is configured to receive the emitted light F2 from the second sensor layer 240b from the plurality of sensor layers 240.

In some embodiments, the optical filter 245 is disposed between the first fiber 204 and the second fiber 216. Specifically, in the illustrated embodiment of FIG. 12, each of the first optical filter 245a and the second optical filter 245b is disposed between the first fiber 204 and the second fiber 216.

The arrangement and configuration of the plurality of light emitting regions 212, the plurality of sensor layers 240, the plurality of optical filters 245, and the plurality of light receiving regions 224 of the dressing system 292 may allow the processor 226 (shown in FIG. 1) to determine an average value of the one or more parameters at the monitoring site. Therefore, the dressing system 292 may improve an accuracy of sensing and monitoring of the analyte by reducing anomalous values that may occur in single point measurements.

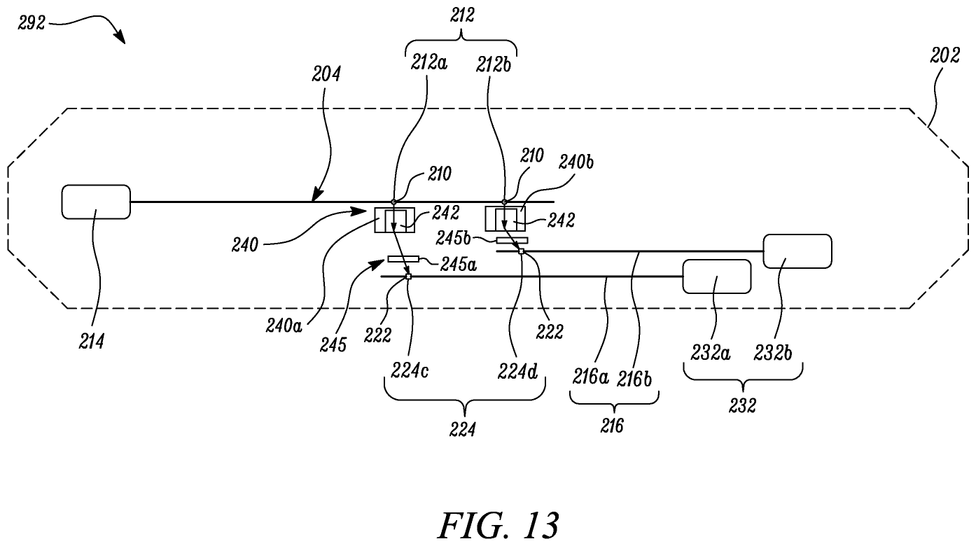

FIG. 13 illustrates a dressing system 292 for sensing a presence of an analyte according to an embodiment of the present disclosure. The dressing system 292 is similar to the dressing system 290 of FIG. 12, with like elements designated by like numbers. However, the dressing system 292 has a different configuration of the second fiber 216 as compared to the dressing system 290. Further, the dressing system 292 has a different configuration of the light detector 232 as compared to the dressing system 290. Some elements of the dressing system 292 are not shown in FIG. 13 for illustrative purposes.

In some embodiments, the second fiber 216 includes a plurality of second fibers 216. Specifically, in the illustrated embodiment of FIG. 13, the plurality of second fibers 216 includes a second fiber 216a and a second fiber 216b. However, it may be noted that the plurality of second fibers 216 may include any number of the second fibers 216, as per desired application attributes.

In the illustrated embodiment of FIG. 13, the plurality of light emitting regions 212 includes the first light emitting region 212a and the second light emitting region 212b. Each of the plurality of light emitting regions 212 is depicted by a circle in FIG. 13.

Further, in the illustrated embodiment of FIG. 13, the plurality of second fibers 216 includes a plurality of light receiving regions 224. Each of the plurality of light receiving regions 224 is depicted by a square in FIG. 13.

Specifically, in the illustrated embodiment of FIG. 13, the plurality of light receiving regions 224 includes a first light receiving region 224c and a second light receiving region 224d. More specifically, in the illustrated embodiment of FIG. 13, the second fiber 216a includes the first light receiving region 224c and the second fiber 216b includes the second light receiving region 224b.

In the illustrated embodiment of FIG. 13, the plurality of optical filters 245 includes the first optical filter 245a and the second optical filter 245b. In the illustrated embodiment of FIG. 13, the first optical filter 245a is disposed between the first fiber 204 and the second fiber 216a proximal to the first light receiving region 224a. Further, in the illustrated embodiment of FIG. 13, the second optical filter 245b is disposed between the first fiber 204 and the second fiber 216b proximal to the second light receiving region 224b.

Moreover, in the illustrated embodiment of FIG. 13, the first sensor layer 240a is disposed proximal to the first optical filter 245a, and between the first light emitting region 212a of the first fiber 204 and the first light receiving region 224a of the second fiber 216a. Further, in the illustrated embodiment of FIG. 13, the second sensor layer 240b is disposed proximal to the second optical filter 245b, and between the second light emitting region 212b of the first fiber 204 and the second light receiving region 224b of the second fiber 216b. Moreover, the first optical filter 245a is spaced apart from the first sensor layer 240a and the second optical filter 245b is spaced apart from the second sensor layer 240b.

In some embodiments, the light detector 232 includes a plurality of light detectors 232. Specifically, in the illustrated embodiment of FIG. 13, the plurality of light detectors 232 includes a first light detector 232a and a second light detector 232b. However, it may be noted that the plurality of light detectors 232 may include any number of the light detectors 232, as per desired application attributes and corresponding to a number of the second fibers 216. In the illustrated embodiment of FIG. 13, the first light detector 232a is electrically coupled to the second fiber 216a and the second light detector 232b is electrically coupled to the second fiber 216b.

The first light detector 232a may be used to determine the one or more parameters at a first location at the monitoring site, and the second light detector 232b may be used to determine the one or more parameters at a second location different from the first location of the monitoring site.

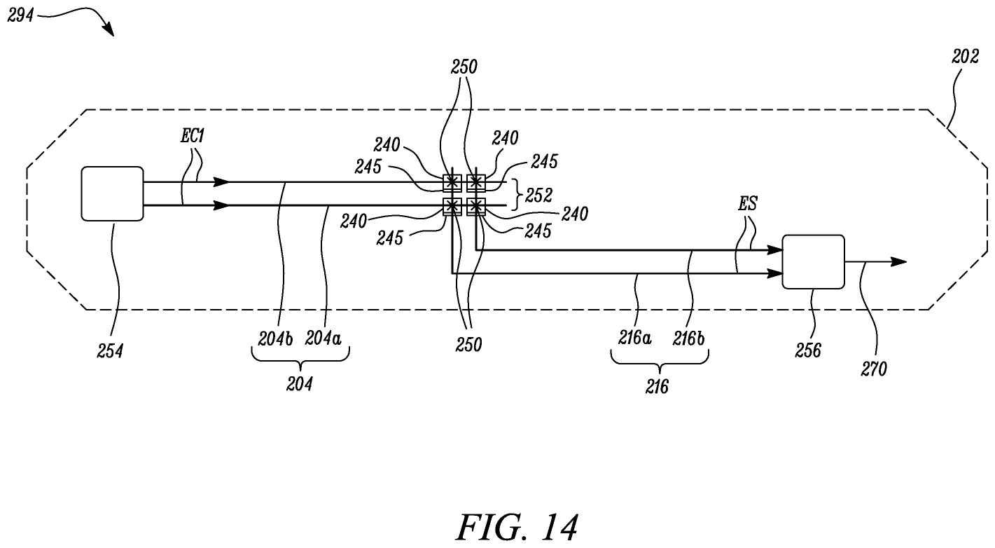

FIG. 14 illustrates a dressing system 294 for sensing a presence of an analyte according to an embodiment of the present disclosure. The dressing system 294 is similar to the dressing system 200 of FIG. 9, with like elements designated by like numbers. However, the dressing system 294 has a different arrangement of the first fiber 112 and the second fiber 126 as compared to the dressing system 200. Further, the dressing system 294 has a different arrangement of the sensor layer 240 and the optical filter 245 as compared to the dressing system 200. Some elements of the dressing system 294 are not shown in FIG. 14 for illustrative purposes.

In the illustrated embodiment of FIG. 14, the first fiber 204 includes a plurality of first fibers 204. Specifically, in the illustrated embodiment of FIG. 14, the plurality of first fibers 204 includes a first fiber 204a and a first fiber 204b. However, it may be noted that the plurality of first fibers 204 may include any number of the first fibers 204, as per desired application attributes. Furthermore, in the illustrated embodiment of FIG. 14, the plurality of second fibers 216 includes the second fiber 216a and the second fiber 216b. However, it may be noted that the plurality of second fibers 216 may include any number of the second fibers 216, as per desired application attributes. In the illustrated embodiment of FIG. 14, a number of the second fibers 216 is equal to a number of the first fibers 204.

In the illustrated embodiment of FIG. 14, the plurality of first fibers 204 and the plurality of second fibers 216 form N×M discrete regions 250 (where N=a number of the first fibers 204 and M=the number of the second fibers 216). Each of the N×M discrete regions 250 is depicted by a cross in FIG. 14. In some embodiments, the N×M discrete regions 250 are formed at a plurality of intersections between the plurality of first fibers 204 and the plurality of second fibers 216.

Further, in the illustrated embodiment of FIG. 14, the plurality of first fibers 204 and the plurality of second fibers 216 are multiplexed to form a N×M fiber grid 252 such that the N×M fiber grid 252 monitors the N×M discrete regions 250 formed at the plurality of intersections between the plurality of first fibers 204 and the plurality of second fibers 216. In other words, in the illustrated embodiment of FIG. 14, the N×M discrete regions 250 may be discrete areas of the monitoring site that may be monitored by the dressing system 294.

In the illustrated embodiment of FIG. 14, the sensor layer 240 includes the plurality of sensor layers 240 and the optical filter 245 includes the plurality of optical filters 245. Further, in the illustrated embodiment of FIG. 14, each of the plurality of sensor layers 240 and each of the plurality of optical filters 245 are disposed in a corresponding discrete region 250 from the N×M discrete regions 250.

In some embodiments, the dressing system 294 includes a multiplexed current source 254 electrically coupled to the plurality of first fibers 204. In some embodiments, the multiplexed current source 254 is configured to supply the electrical current EC1 to each of the plurality of first fibers 204. In some embodiments, the multiplexed current source 254 may be configured to selectively supply the electrical current EC1 to one or more of the plurality of first fibers 204.

In some embodiments, the dressing system 294 further includes a multiplexed light detector 256 electrically coupled to the plurality of second fibers 216. In some embodiments, the multiplexed light detector 256 is configured to generate the input signal 270 in response to the electrical signal ES. The multiplexed light detector 256 may include an ammeter, a multimeter, an oscilloscope, and the like. As discussed above, in some embodiments, the processor 226 (shown in FIG. 9) is further configured to generate the output signal 272 (shown in FIG. 9) indicative of the one or more parameters based on the input signal 270.

Therefore, the dressing system 294 may utilize the multiplexed current source 254, the multiplexed light detector 256, and the N×M fiber grid 252 for sensing and monitoring of the analyte at each of the N×M discrete regions 250.

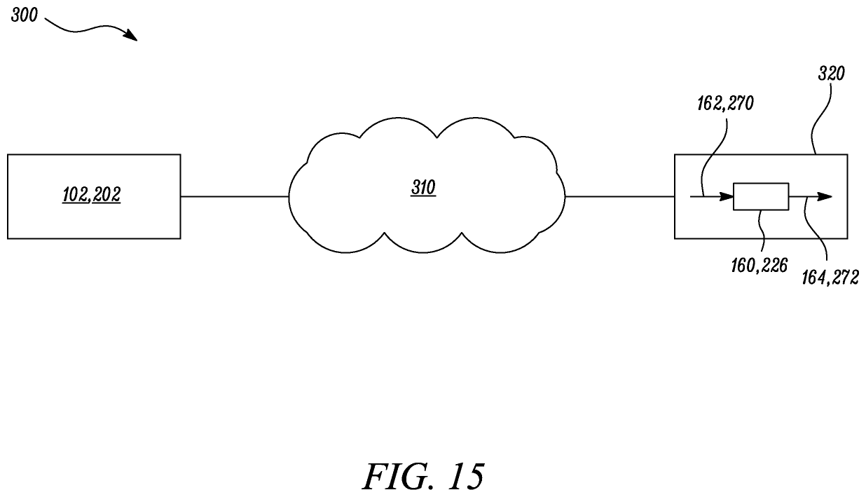

FIG. 15 illustrates a dressing system 300 for sensing a presence of an analyte according to an embodiment of the present disclosure.

In illustrated embodiment of FIG. 15, the dressing system 300 includes the dressings 102, 202 and the processors 160, 226. Specifically, in the illustrated embodiment of FIG. 15, the processors 160, 226 are disposed in a computing device 320. However, in some other embodiments, the processors 160, 226 may be remotely operated standalone devices communicably coupled to the dressings 102, 202. In some embodiments, the computing device 320 may include, but is not limited to, a personal computer, a laptop computer, a cellular telephone, a smartphone, a wearable device, a tablet computer, and so forth.

In the illustrated embodiment of FIG. 15, the dressing system 300 further includes a network 310 (e.g., a local area network) via which the dressings 102, 202 communicate with the computing device 320.

In some examples, the network 310 may include one or more wireless networks, a wired network, a local area network (LAN), a metropolitan area network (MAN), a wide area network (WAN), a wireless personal area network (WPAN), WiMax networks, a direct connection, such as through a Universal Serial Bus (USB) port, and/or the like, and may include a set of interconnected networks that make up the Internet. In some examples, the wireless network may include a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc. In some examples, the network 310 may include a circuit-switched voice network, a packet-switched data network, or any other network capable for carrying electronic communication. For example, the network 310 may include networks based on the Internet protocol (IP) or asynchronous transfer mode (ATM), etc. Examples of the network 310 may further include, but are not limited to, a personal area network (PAN), a storage area network (SAN), a home area network (HAN), a campus area network (CAN), an enterprise private network (EPN), the Internet, a global area network (GAN), and so forth.

In some examples, the dressings 102, 202 may include various components, such as a communication module (not shown), mounted thereon or otherwise accessible to the dressings 102, 202. Specifically, the dressings 102, 202 may transmit information through the communication module. In some embodiments, the computing device 320 may include associated wired/wireless communication interface for communicating with the dressings 102, 202.

Referring to FIGS. 1 and 15, as discussed above, in some embodiments, the processor 160 is communicably coupled to the light detector 136. In some embodiments, the light detector 136 may communicate with the processor 160 via the network 310. Therefore, in some embodiments, the input signal 162 generated by the light detector 136 may be transmitted to the processor 160 via the network 310. In some embodiments, the processor 160 is further configured to generate the output signal 164 indicative of the one or more parameters based on the input signal 162.

Referring to FIGS. 9 and 15, as discussed above, in some embodiments, the processor 226 is communicably coupled to the light detector 232. In some embodiments, the processor 226 is communicably coupled to the light detector 232 and the multiplexed light detector 256 (shown in FIG. 14). In some embodiments, the light detector 232 and the multiplexed light detector 256 may communicate with the processor 226 via the network 310. Therefore, in some embodiments, the input signal 270 generated by the light detector 232 or the multiplexed light detector 256 may be transmitted to the processor 226 via the network 310. In some embodiments, the processor 226 is further configured to generate the output signal 272 indicative of the one or more parameters based on the input signal 270.

In some embodiments, the processors 160, 226 may be configured to execute a set of computer executable instructions to generate the output signals 164, 272 based on the received input signals 162, 270 to determine a numerical value of each of the one or more parameters. In some embodiments, the one or more parameters are at least one of an oxygen concentration, a blood pressure, a temperature, a pH value, a glucose level, and an infection status. In some embodiments, the processors 160, 226 may be further configured to display the determined numerical value of each of the one or more parameters using a display interface (not shown) of the computing device 320.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A dressing system for sensing a presence of an analyte, the dressing system comprising:
a first layer comprising a first major surface and a second major surface opposite to the first major surface, wherein the first layer has a first permeability to the analyte;
a second layer facing the first major surface of the first layer, wherein the second layer has a second permeability to the analyte less than the first permeability of the first layer;
a first fiber at least partially disposed between the first layer and the second layer, wherein the first fiber is configured to deliver an excitation light;
at least one sensor layer comprising a sensor material disposed between the first layer and the second layer, wherein the at least one sensor layer is configured to receive the excitation light from the first fiber and emit an emitted light in response to the excitation light, wherein the emitted light comprises a first optical property sensitive to the presence of the analyte; and
a second fiber separate from the first fiber and disposed between the first layer and the second layer, wherein the second fiber is configured to receive the emitted light from the at least one sensor layer.

2. The dressing system of claim 1, wherein the first permeability is greater than the second permeability by a factor of at least 2.

3. The dressing system of claim 1, further comprising an adhesive layer disposed on the second major surface of the first layer and comprising an adhesive material.

4. The dressing system of claim 3, wherein the adhesive layer has a third permeability to the analyte greater than or equal to the first permeability of the first layer.

5. The dressing system of claim 3, wherein the adhesive layer has a third permeability to the analyte, and wherein the third permeability is greater than the second permeability by a factor of at least 1.5.

6. The dressing system of claim 1, further comprising a light source optically coupled to the first fiber and configured to emit the excitation light.

7. The dressing system of claim 1, wherein the first fiber comprises at least one light emitting region disposed between the first layer and the second layer and configured to emit the excitation light.

8. The dressing system of claim 1, wherein the first fiber comprises a fiber body, at least one electrical conductor disposed within the fiber body and extending at least partially along a length of the fiber body, and at least one light emitting device electrically connected to the at least one electrical conductor and configured to emit the excitation light in response to an electrical current.

9. The dressing system of claim 8, further comprising a current source electrically coupled to the at least one electrical conductor and configured to supply the electrical current to the at least one light emitting device via the at least one electrical conductor.

10. The dressing system of claim 9, wherein the current source is at least one of a battery, a capacitor, a wearable nanogenerator, and a wireless charging circuit.

11. The dressing system of claim 1, wherein the second fiber comprises at least one light receiving region disposed between the first layer and the second layer and configured to receive the emitted light from the at least one sensor layer.

12. The dressing system of claim 1, further comprising a light detector optically or electrically coupled to the second fiber and configured to receive an optical signal or an electrical signal corresponding to the emitted light.

13. The dressing system of claim 12, wherein the light detector comprises at least one of a photodetector, a camera, a spectrometer, a multimeter, and an oscilloscope.

14. The dressing system of claim 1, wherein the first optical property is at least one of an optical intensity of the emitted light, a photoluminescence lifetime of the emitted light, and a wavelength of the emitted light.

15. The dressing system of claim 1, wherein the emitted light comprises the first optical property having a first value in an absence of the analyte and the emitted light comprises the first optical property having a second value in the presence of the analyte, and wherein the first value is different from the second value.

16. The dressing system of claim 1, wherein the excitation light comprises a second optical property different from the first optical property and having a third value, and wherein the emitted light comprises the second optical property having a fourth value different from the third value.

17. The dressing system of claim 16, further comprising at least one optical filter configured to receive the emitted light from the at least one sensor layer, wherein the at least one optical filter comprises a third optical property having respective fifth and sixth values in response to the third and fourth values of the second optical property, and wherein the sixth value is greater the fifth value by a factor of at least 2.

18. The dressing system of claim 17, wherein the second fiber comprises a fiber tip at one end of the second fiber, wherein the at least one optical filter is disposed on the fiber tip of the second fiber.

19. The dressing system of claim 17, wherein the at least one optical filter is disposed proximal to an outer surface of the second fiber.

20. The dressing system of claim 17, wherein the at least one optical filter is at least partially disposed within the second fiber.

21-48. (canceled)
